SEQUENCE LISTING (22 sequences)

SEQ ID NO: 1 - protein, 21 aa, Artificial Sequence
  Residues l3l-lSl of a spliceosome protein
RIHMVYSKRSGKPRGYAFIEY

SEQ ID NO: 2 - DNA, 2715 bp, Artificial Sequence
  SMI41 sequence in AAV
atggcgacgggttcaagaacttccctacttcttgcatttggcctgctttgtttgccgtggttacaggagggctcggcacaagttactctaaaagagtctggccctgggatattgaggccctcacagaccctcgatctgacttgttctttctctgggttttcactgagcacttctggtctgagtgtaggctggattcgtcagccttcagggaagggtctggagtggctggcacacatttggtgggatgatgtgaagtactttaacccatccctgaagagcagactcacaatctccaaggatagctccagaaaccaggtgttcctcaagatcaccagtgtggacactgcagatagtgccacataccactgtactcgaggacctctgggtcacggatttgactactggggccaagggactctggtcactgtctctgccgctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccgcaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcagatgttgtgatgacccaaactccactctccctgcctgtcagtcttggagatcaagccttcatctcttgcagatctagtcagagccttgtacacagtgatggaaacagctacttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggactttatttctgctctcaaactacacatgttccttggacgttcggtggaggcaccaagctggaaatcaaacgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtcacggatgttctgagcccctgggcctgaagaataacacaattcctgacagccagatgtcagcctccagcagctacaagacatggaacctgcgtgcttttggctggtacccccacttgggaaggctggataatcagggcaagatcaatgcctggacggctcagagcaacagtgccaaggaatggctgcaggttgacctgggcactcagaggcaagtgacaggaatcatcacccagggggcccgtgactttggccacatccagtatgtggcgtcctacaaggtagcccacagtgatgatggtgtgcagtggactgtatatgaggagcaaggaagcagcaaggtcttccagggcaacttggacaacaactcccacaagaagaacatcttcgagaaacccttcatggctcgctacgtgcgtgtccttccagtgtcctggcataaccgcatcaccctgcgcctggagctgctgggctgttaa

SEQ ID NO: 3 - protein, 662 aa, Artificial Sequence
  SMI41 sequence in AAV
QVTLKESGPGILRPSQTLDLTCSFSGFSLSTSGLSVGWIRQPSGKGLEWLAHIWWDDVKYFNPSLKSRLTISKDSSRNQVFLKITSVDTADSATYHCTRGPLGHGFDYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKDVVMTQTPLSLPVSLGDQAFISCRSSQSLVHSDGNSYLHWYLQKPGQSPKLLIYKVSNRFSGVPDRFSGSGSGTDFTLKISRVEAEDLGLYFCSQTTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 4 - DNA, 741 bp, Artificial Sequence
  Phosphatidylserine-binding peptide
atggcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaaccacggatgttctgagcccctgggcctgaagaataacacaattcctgacagccagatgtcagcctccagcagctacaagacatggaacctgcgtgcttttggctggtacccccacttgggaaggctggataatcagggcaagatcaatgcctggacggctcagagcaacagtgccaaggaatggctgcaggttgacctgggcactcagaggcaagtgacaggaatcatcacccagggggcccgtgactttggccacatccagtatgtggcgtcctacaaggtagcccacagtgatgatggtgtgcagtggactgtatatgaggagcaaggaagcagcaaggtcttccagggcaacttggacaacaactcccacaagaagaacatcttcgagaaacccttcatggctcgctacgtgcgtgtccttccagtgtcctggcataaccgcatcaccctgcgcctggagctgctgggctgttaa

SEQ ID NO: 5 - protein, 246 aa, Artificial Sequence
  Phosphatidylserine-binding peptide
MAFVNQHLCGSHLVEALYLVCGERGFFYTPKTRREAEDLQVGQVELGGGPGAGSLQPLALEGSLQKRGIVEQCCTSICSLYQLENYCNHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 6 - protein, 8 aa, Artificial Sequence
  Phosphatidylserine-binding peptide
LSYYPSYC

SEQ ID NO: 7 - protein, 12 aa, Artificial Sequence
  Phosphatidylserine-binding peptide
AREDGYDGAMDY

SEQ ID NO: 8 - protein, 6 aa, Artificial Sequence
  Phosphatidylserine-binding peptide
LIKKPF

SEQ ID NO: 9 - protein, 14 aa, Artificial Sequence
  Phosphatidylserine-binding peptide
CLIKKPFCLIKKPF

SEQ ID NO: 10 - protein, 6 aa, Artificial Sequence
  Phosphatidylserine-binding peptide
PGDLSR

SEQ ID NO: 11 - protein, 7 aa, Artificial Sequence
  Phosphatidylserine-binding peptide
CPGDLSR

SEQ ID NO: 12 - protein, 14 aa, Artificial Sequence
  Phosphatidylserine-binding peptide
FNFRLKAGQKIRFG

SEQ ID NO: 13 - protein, 14 aa, Artificial Sequence
  Phosphatidylserine-binding peptide
FNFRLKAGAKIRFG

SEQ ID NO: 14 - protein, 14 aa, Artificial Sequence
  Phosphatidylserine-binding peptide
FNFRLKVGAKIRFG

SEQ ID NO: 15 - protein, 14 aa, Artificial Sequence
  Phosphatidylserine-binding peptide
FNFRLKTGAKIRFG

SEQ ID NO: 16 - protein, 14 aa, Artificial Sequence
  Phosphatidylserine-binding peptide
FNFRLKCGAKIRFG

SEQ ID NO: 17 - protein, 12 aa, Artificial Sequence
  Phosphatidylserine-binding peptide
RSRRMTRRARAA

SEQ ID NO: 18 - protein, 6 aa, Artificial Sequence
  Phosphatidylserine-binding peptide
TLVSSL

SEQ ID NO: 19 - protein, 37 aa, Artificial Sequence
  Phosphatidylserine-binding peptide
TRYLRIHPRSWVHQIALRLRYLRIHPRSWVHQIALRS

SEQ ID NO: 20 - protein, 37 aa, Artificial Sequence
  Phosphatidylserine-binding peptide
TRYLRLHPRSWVHQLALRLRYLRLHPRSWVHQLALRS

SEQ ID NO: 21 - protein, 24 aa, Artificial Sequence
  Phosphatidylserine-binding peptide
KKKKRFSFKKSFKLSGFSFKKNKK

SEQ ID NO: 22 - protein, 9 aa, Artificial Sequence
  Phosphatidylserine-binding peptide
CLSYYPSYC

(19) United States
(12) Patent Application Publication
(10) Pub. No.: US 2016/0060358 A1
(43) Pub. Date: Mar. 3, 2016

(54) INDUCTION OF ANTIGEN-SPECIFIC TOLERANCE

(71) Applicant: CALIFORNIA INSTITUTE OF TECHNOLOGY, Pasadena, CA (US)

(72) Inventor: Bruce A. Hay, Encino, CA (US)

(21) Appl. No.: 14/837,941

(22) Filed: Aug. 27, 2015

Related U.S. Application Data
(60) Provisional application No. 62/042,888, filed on Aug. 28, 2014.

(51) Int. Cl. C07K 16/42; C07K 14/00; C07K 16/26; C07K 14/755; C07K 14/62; C07K 14/78; C07K 14/435; C07K 14/005; C12N 7/00; C07K 14/72; C12N 9/08; C07K 14/745

(52) U.S. Cl. CPC C07K 16/42; C12N 9/0065; C07K 14/00; C07K 16/26; C07K 14/755; C07K 14/745; C07K 14/78; C07K 14/435; C07K 14/005; C12N 7/00; C07K 14/723; C07K 14/62; C12N 2730/10122; C12N 2730/10134; A61K 39/00

(57) ABSTRACT

Described are compositions and methods for the induction of an antigen-specific tolerance in a vertebrate. Also described are compositions and methods for the induction of antigen-specific tolerance using a fusion or a complex of the antigen (e.g., an antibody or an enzyme) against which tolerance is desired with a phosphatidylserine-binding domain derived from a phosphatidylserine-binding protein (including peptides).

### PRIORITY

This application claims the benefit of U.S. provisional application 62/042,888 filed on Aug. 28, 2014, which is hereby incorporated by reference in its entirety.

### REFERENCE TO SEQUENCE LISTING IN ELECTRONIC FORMAT

The present application is being filed along with a sequence listing in electronic format. The sequence listing is provided as a file entitled CALTE111A_Sequence_Listing.TXT which is 16,902 bytes in size, created on Aug. 26, 2015 and last modified on Aug. 26, 2015. The information in the electronic format of the Sequence Listing is incorporated herein by reference in its entirety.

### BACKGROUND

1. Field

The present disclosure generally relates to compositions and methods for induction of antigen-specific tolerance.

2. Description of the Related Art

Until recently, therapeutic induction of tolerance relied on broad-based approaches that resulted in cellular depletion of all B and/or T cells or cytokine profile alteration. These broad-based approaches weaken the immune system in general and leave many subjects vulnerable to opportunistic infections, autoimmune attack and cancer. One set of antigen-specific approaches is difficult, and includes introduction of autologous tolerogenic dendritic cells expressing protein to which tolerance is desired, such as recombinant FVIII, or introduction of apoptotic cells from the patient in association with the antigen of interest.

A second set of approaches involves loading of RBCs with antigens and delivery through IV injection. This can result in tolerance to otherwise highly immunogenic proteins, such as ovalbumin (Cremel et al., 2013). In a related approach, proteins to which tolerance is desired can be coupled to an erythrocyte-binding peptide or an erythrocyte binding antibody (Kontos et al., 2013). IV injection of these proteins resulted in tolerance induction in mice.

Finally, it has also been shown that formation of complexes between blood clotting factors such as factor FVIII and phosphatidylserine (PS) resulted in a reduced antibody response to FVIII in mice (Purohit et al., 2005; Ramani et al., 2008) (Gaitonde et al., 2011). This was shown to be tolerogenic in that it could be adoptively transferred to naive mice (Gaitonde et al., 2013). This approach uses PS to target the antigen of interest to cells that will present it in a tolerogenic manner.

### SUMMARY

In some embodiments, a tolerance-inducing molecule is provided that comprises an antigen, and a phosophatidylserine-binding protein associated with the antigen to form an antigen-phosophatidylserine-binding protein fusion (“APBP”) and/or an antigen-phosophatidylserine-binding protein complex (“APBC”).

In some embodiments, a nucleic acid sequence encoding any one or more of the tolerance-inducing molecules provided herein.

In some embodiments, a vector comprising the nucleic acid sequence of any of the embodiments provided herein is provided.

In some embodiments, a composition is provided. The composition comprises a mixture of any one of the tolerance-inducing molecules provided herein and a free therapeutic molecule. The free therapeutic molecule is not associated with the antigen.

In some embodiments, a method of providing immunological tolerance to an antigen is provided. The method comprises administering an effective amount of a tolerance-inducing molecule, the tolerance-inducing molecule comprises a phosophatidylserine-binding protein that is associated with an antigen to a subject.

### BRIEF DESCRIPTION OF THE DRAWINGS

FIG. 1 schematizes embodiments of an antibody containing a PSBD in various positions.

FIG. 2A illustrates the nucleotide sequence of an embodiment of a mouse antibody designed to bind GnRH.

FIG. 2B illustrates the translated protein sequence of the nucleotide sequence of the mouse antibody of FIG. 2A designed to bind GnRH.

FIG. 3 illustrates the nucleotide and translated protein sequence of an embodiment of human prepro-insulin and the C2 domain of lactadherin.

FIG. 4 is a schematic of an embodiment of an adeno-associated virus (AAV) vector for expression of an antigen-PSBD fusion.

### DETAILED DESCRIPTION

Antigen-specific tolerance is desired in autoimmunity, transplantation allergy and other diseases, and is also desirable in the context of therapy with autologous proteins and non-autologous proteins. However, there is also value for a less aggressive and a more targeted approach to the induction of immune tolerance. For example, an approach that can be implemented in long-term formats and that does not require the patient to come in for repeated treatments could be very useful for some applications.

Such a method can be especially useful for those receiving recombinant proteins. There are a variety of recombinant proteins (RP) that are introduced into people on a chronic basis. Adverse reactions occur in some of these patients. In addition, induction of an anti-drug immune response can result in loss of RP efficacy. Antibodies generated against the RP are one important mechanism by which the abovementioned failures can occur. In some cases the RP is a foreign protein, and the RP is simply seen as non-self and eliminated through activation of an immune response. In other cases, antibodies are raised against therapeutic antibodies, which have undergone extensive “humanization” so as to be rendered as “self like” as possible. However, even in these cases anti-antibody responses are sometimes induced. These can arise in two different ways. First, within the human population there are polymorphisms for sequences of the constant regions. These differences (allotypes) can be recognized as foreign, resulting in the induction of antibodies directed against the therapeutic antibody (Jefferis and Lefranc, 2009; Pandey and Li, 2013). Second, each antibody has a unique set of variable domain sequences. These idiotypes can also be recognized as foreign, resulting in the creation of anti-idiotypic antibodies. Both sorts of responses have the same effect, to neutralize the function of the therapeutic antibody. Similar problems can arise in the context of other therapeutics as well. For example, some hemophiliacs lack factor VIII, factor IX, or factor XI. These can be provided in recombinant form. However, in some fraction of patients an immune response develops, resulting in the appearance of inhibitory antibodies. In each of the above cases, parts of a protein are being recognized as foreign, even if much of the rest of the molecule has sequence identity with abundant self-proteins.

In some embodiments, the methods and compositions provided herein allow one to create versions of a recombinant protein that prevent and/or reduce the induction of an immune response targeting that protein. Thus, antigen-specific tolerance for specific therapeutics can be brought about. In some embodiments, this is achieved by tagging the protein of interest with a peptide or protein domain that has an affinity for the lipid phosphatidylserine (PS). As detailed further below, PS is exposed on the surface cells dying through the process of apoptosis. Apoptotic cells are engulfed by macrophages, and many other cell types. Apoptotic cells are tolerogenic with respect to antigens they express. Therefore, because the therapeutic is tagged with a PS-binding domain, it will be taken up with the dying cell, resulting in tolerance to the therapeutic, as with other proteins expressed by the apoptotic cell.

Thus, in some embodiments, by the compositions and methods provided herein, one can induce long-term antigen-specific immune tolerance. The embodiments described herein bring this about in the following way. Antigens to which an immune response is not desired (antigens to which one wants to induce and/or maintain tolerance) are linked covalently or non-covalently to peptides or proteins that have a high affinity for the membrane lipid phosphatidylserine (PS). PS is exposed on the surface of cells undergoing apoptosis. The presence of the PS-binding domain results in the binding of some amount of the antigen to the surface of the dying cell. More than 200 billion cells die through apoptosis each day. These are phagocytosed by cells of the spleen, liver and other tissues. Antigens bound to these dying cells are taken up by phagocytosis along with the dying cell. Macrophages and other cells that take up these cells present their proteins to other cells of the immune system. In general the proteins taken up from cells undergoing apoptosis are presented in a way that induces tolerance to them (the antigens) rather than activation of an immune response. Without intending to be limited by theory, this makes sense because these are proteins that are a normal part of the body and the body wants to make sure that it avoids creating aberrant immune responses to self-proteins. Presentation of antigens from normally occurring apoptotic cells in a way that inhibits activation of immune cells that recognize these proteins is a primary way in which tolerance to self is brought about. This is further supported by the fact that the elimination of PS-dependent uptake of dying cells, or removal of the phagocytic cells, results in autoimmune disease. In addition, loading of antigens into or onto apoptotic cells and then infusing them into an individual IV results in tolerance to the antigen.

In some embodiments, compositions and methods for induction of antigen-specific tolerance using antigen-phosphatidylserine-binding protein fusions or complexes are provided.

### DEFINITIONS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive. Unless defined otherwise, technical and scientific terms used herein have the same meaning as commonly understood by one of ordinary skill in the art. See, for example Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N. Y. 1989). For purposes of the present disclosure, the following terms are defined below. It is to be understood that both the foregoing general description and the following detailed description are exemplary and explanatory only and are not intended to be restrictive. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As defined herein, a “tolerance-inducing molecule” (which may also be referred to as the “tolerance molecule”) is a complex of an antigen with either a PS-binding domain or a PS-binding protein. The tolerance-inducing molecule, when introduced into a host, such as human or animal, induces immunological tolerance against the antigen in the host.

Phosphatidylserine (PS) is an important phospholipid of the cell membranes which plays a key role in cell cycle signaling, specifically in apoptosis. Phosphatidylserine is biosynthesized in bacteria by condensing the amino acid serine with CDP (cytidine diphosphate)-activated phosphatidic acid. In mammals, phosphatidylserine is produced by base-exchange reactions with phosphatidylcholine and phosphatidylethanolamine. Conversely, phosphatidylserine can also give rise to phosphatidylethanolamine and phosphatidylcholine, although in animals the pathway to generate phosphatidylcholine from phosphatidylserine only operates in the liver.

As used herein, a “PS-binding domain” is a polypeptide domain, which can be a or part of a protein that binds to PS, including shorter peptides (for example, 6-25 amino acids in length). The PS-binding domain can be derived from a protein or can be synthetic. The binding domain can be a full length protein or peptide and/or a fragment thereof.

As used herein, a “PS-binding protein (PSBP)” is a protein that has one or more domains that binds PS or a polypeptide fragment of which binds PS.

As used herein, an “antigen-phosophatidylserine-binding protein fusion (“APBP”) and/or an antigen-phosophatidylserine-binding protein complex (“APBC”)” is a complex of an antigen that is coupled, either covalently or non-covalently to a PS-binding domain or a PS-binding protein

As used herein, an “antigen” refers to a therapeutic molecule, polypeptide or protein against which an immune response is raised either normally leading to immunological clearance of the antigen or abnormally leading to generation of an uncontrolled immunological response or autoimmune response, which may lead to destruction of the antigen-expressing tissue.

As used herein, a “therapeutic molecule” is a protein or non-protein molecule that is used to attain a therapeutic effect such as a reduction in an unwanted immune response or prevention of the occurrence of such a response.

As used herein, “free therapeutic molecule” refers to a therapeutic molecule that is not conjugated to PS-binding domain or PS-binding protein. In some embodiments, it can be administered separately from the tolerance-inducing molecule. In some embodiments, the free therapeutic molecule is administered subsequent to the administration of the tolerance-inducing molecule. In some embodiments, the free therapeutic molecule can be administered with the tolerance-inducing molecule. In some embodiments the free therapeutic molecule refers to a protein normally synthesized by the body in some condition or other; in other words, the free therapeutic molecule is not administered to the subject in all embodiments.

As used herein, “protein” refers to the macromolecule comprising one or more polypeptide chain of amino acids. The polypeptide chains can be covalently or non-covalently linked to each other. The one or more of the polypeptide chains can have modifications such as glycosylation, phosphorylation, etc.

As used herein, “covalently linked” refers to linkage by a covalent bond, which is a chemical bond that involves the sharing of electron pairs between atoms.

As used herein, “non-covalently linked” refers to linkage by a non-covalent interaction which, unlike a covalent bond, does not involve the sharing of electrons, but rather involves more dispersed variations of electromagnetic interactions between molecules or within a molecule. Non-covalent interactions can be generally classified into four categories: electrostatic, π-effects, van der Waals forces, and hydrophobic effects.

As used herein, the term “vector” refers to a polynucleotide construct, used to transmit genetic material to a host cell. Vectors can be DNA- or RNA-based. DNA-based vectors can be non-viral, and include molecules such as plasmids, minicircles, closed linear DNA, doggybones, linear DNA, and single-stranded DNA (Yin et al., 2014). DNA-based vectors can also be viral, and include adeno-associated virus, lentivirus, adenovirus, and others (Kay, 2011). Vectors can also be RNA. These can be linear or circular forms of unmodified RNA (e.g. (Wang and Wang, 2015)). They can also include various nucleotide modifications designed to increase half-life, decrease immunogenicity, and/or increase level of translation (Youn and Chung, 2015). A vector as used herein can be composed of either DNA or RNA. In some embodiments, a vector is composed of DNA. Vectors are preferably capable of autonomous replication in a prokaryote such as E. coli, used for growth. In some embodiments the vector may be stably integrated into the genome of the organism of interest. In many others the vector remains separate, either in the cytoplasm or the nucleus (Kay, 2011 {Yin, 2014 #4837). In some embodiments, a vector contains a targeting sequence. In some embodiments, the vector comprises (contains) an antibiotic resistance gene. The vector comprises regulatory elements for regulating gene expression.

As used herein, “associated virus (AAV) vector” refers to the virus that is widely used for generating viral vectors for therapeutic interventions such as gene therapy as well as for gene expression.

As used herein, “inverted terminal repeat (ITR)” refers to a nucleic acid sequence that when located flanking a second nucleic acid sequence enables transfer of the second nucleic acid sequence within and/or between one or more genomes, and/or promotes replication—in the appropriate intracellular environment—of the DNA located between the ITRs.

The term “regulatory sequence” is used herein to refer to nucleic acid elements that can influence the expression of a coding sequence (for example, a gene) in a particular host organism. These terms are used broadly and cover all elements that promote or regulate transcription, including promoters, core elements required for basic interaction of RNA polymerase and transcription factors, upstream elements, enhancers, and response elements (see, for example, Lewin, “Genes V” (Oxford University Press, Oxford) pages 847-873).

As used here in, “T cells” refers to a type of immune cells that contribute to cellular immunity against an antigen. Several T cell types are known such as CD4 T cells, CD8 T cells, regulatory T cells among others. “B cells” refers to a second type of immune cell which can also contribute to an immune response directed against a specific antigen, often through the expression of immunoglobulins that bind the antigen. Other forms of B cells, regulatory B cells, can play a suppressive role in the immune system (Goode et al., 2014; Wang and Zheng, 2013)

As used herein, “reduce” or “reduced” or “reduction” refers to a decrease in a metric from a first level to a second level. For example, an immune response against an antigen can be reduced, as for example determined by the level of activation of an immune cell population directed against that antigen. The reduction from the first level to the second level can be by about 10, 20, 30, 40, 50, 60, 70, 80, 90, 95, 99, 99.9, 99.99, or 100% (with 100% reduction indicating, for example, no immune response).

As used herein, “self-antigen” refers to a protein or non-protein molecule that is generated within a body as part of the body's normal physiology. Normally, immune responses against self-antigens are suppressed by central and/or peripheral tolerance. However, an abnormal immune response against a self-antigen can result in disease conditions such as autoimmunity.

As used herein, “self-tolerance” refers to a state of unresponsiveness of the immune system against a molecule that has the potential to be an immunogenic compound and elicit an immune response.

As used herein, “autoimmune antigen” refers to antigens against which an immune response is initiated in an autoimmune disease such as diabetes.

As used herein, a “protein of interest” refers to a protein and/or protein antigen against which an immunological tolerance is desired or against which an immune response is to be prevented is desired or which can be used as a free therapeutic molecule. This can include therapeutic molecules.

As used herein, “immunological tolerance” refers to the generation of natural central and/or peripheral tolerance against an antigen and/or generation of induced tolerance against an antigen or a protein of interest as a result of which the body's immune system no longer mounts an immune response against that antigen or protein of interest and/or the tissue in which the antigen is expressed.

As used herein, an “immunogenic compound” refers to a compound or molecule that has the capacity to stimulate an immune response when introduced into a host.

As used herein, a “tolerance-inducing molecule” or “tolerance molecule” refers to a complex of an antigen or protein of interest with a PS-binding domain or PS-binding protein, which induces immunological tolerance against the antigen or protein of interest.

As used herein, “effective amount” refers to an amount that brings about a desired effect. The effective amount may vary for a particular composition depending on several parameters including but not limited to the route of administration, the pharmaceutically acceptable carrier, the individual or animal receiving the composition, the disease condition.

As used herein, “long term” refers to greater than one week.

### Immune Tolerance can Involve Elimination of Autoreactive T Cells

To maintain self-tolerance, autoreactive T cells are initially eliminated in the thymus (central tolerance). To complement central tolerance, autoreactive T cells that escape central tolerance are removed in peripheral organs. Peripheral tolerance to self-antigens is thought to be maintained by antigen presenting cells (APCs), including dendritic cells (DCs) localized in peripheral tissues such as the spleen and liver. Resident DCs constantly phagocytose apoptotic cells generated during normal tissue turnover and migrate to draining lymph nodes (LNs) where they induce deletion or anergy of CD4 and CD8 T cells by presentation of cell-associated antigens obtained from cell corpses/debris.

In addition to the peripheral tolerance induced by residual DCs in LNs, phagocytes in the spleen can also present self-antigens derived from circulating dying cells to induce self-tolerance. It has been argued that the immune system promotes tolerance to antigens associated with apoptotic cells because many such cells are generated every day, and they constitute a major form of self-antigens. Early work pointing towards this hypothesis is reviewed in (Steinman et al., 2000) (Steinman and Nussenzweig, 2002). A more recent review, which focuses on the mechanisms by which tolerance is induced to antigens coupled to apoptotic splenocytes makes similar points, with much more mechanistic detail (Getts et al., 2013).

### Tolerance and the Induction of TReg Cells

Initial self/non-self discrimination occurs in the thymus during neonatal development where medullary epithelial cells express specific self-protein epitopes to immature T cells. T cells recognizing self-antigens with high affinity are deleted, but autoreactive T cells with moderate affinity sometimes avoid deletion and can be converted to so called natural regulatory T cells (TReg) cells. These natural TReg cells are exported to the periphery and provide for constant suppression of autoimmunity. Natural regulatory T cells are a critical component of immune regulation and self-tolerance.

A second form of tolerance occurs in the periphery where mature T cells are converted to an ‘adaptive’ TReg phenotype upon activation via their T cell receptor in the presence of IL-I0 and TGF-Beta, usually supplied by bystander T regulatory cells. The possible roles for these ‘adaptive’ TReg cells include dampening immune response following the successful clearance of an invading pathogen to control excessive inflammation as can be caused by an allergic reaction or low level chronic infection, or possibly to facilitate coexistence with beneficial symbiotic bacteria and viruses. ‘Adaptive’ TReg also play a role in managing the life cycle of B cells and the antibodies they produce, and regulatory B cells and the immunoregulators they produce (Goode et al., 2014; Wang and Zheng, 2013).

It was shown many years ago that simple chemical compounds (haptens) coupled with cellular constituents of blood induced hapten-specific tolerance when introduced IV (Landsteiner and Jacobs, 1935). Subsequently it was shown that tolerance could be brought about by coupling antigens to cell membranes (Battisto and Bloom, 1966). These results suggested that there was some role for cellular components in tolerance induction to foreign antigens.

Ethylene carbodiimide (ECDI) was subsequently used as a coupling agent to deliver antigens associated with membranes, resulting in immunity or tolerance depending on the route of administration, with IV administration resulting in tolerance (Miller et al., 1979). Subsequently it was recognized that a critical feature of this system was the fact that ECDI triggers cell death, and it is this secondary effect that is required for tolerance induction (Luo et al., 2008; Turley and Miller, 2007). In parallel, other observations showed directly that tolerance could be induced to antigens associated with dying cells when they were presented to dendritic cells in vivo (Liu et al., 2002). More recent work showed that induction of tolerance required phagocytosis of the dying cell (Sun et al., 2004). This latter work also showed the power of apoptotic cells to induce tolerance in the context of organ transplant. Apoptotic splenocytes from a donor dramatically prolonged the lifetime of a heart transplant into an unrelated recipient. It is thought that tolerance is induced, in various ways, following apoptotic cell uptake by antigen presenting cells (Getts et al., 2013; Getts et al., 2011; Turley and Miller, 2007). Sites of tolerance induced by death generally are reviewed in (Ravishankar and McGaha, 2013). The spleen and its resident macrophages are likely to play an important role, as tolerance to antigen coupled to apoptotic splenocytes is ineffective in splenectomyzed mice and cannot be induced by subcutaneous (SC) or intraperitoneal (IP) administration (Getts et al., 2011). Also, apoptotic cells loaded with an immunogenic antigen induce tolerance to that antigen, but tolerance fails to occur if spleen macrophages are removed (Miyake et al., 2007). Spleen marginal zone macrophages play an important role in uptake and presentation of antigens from apoptotic cells in a toleragenic manner (McGaha et al., 2011; Ravishankar et al., 2012; Ravishankar et al., 2014). Much of this uptake is mediated through a phosphatidylserine (PS)-dependent mechanism.

The liver is likely to also play an important role in induction of tolerance to apoptotic cell-associated antigens because it is a major site for the removal of aging erythrocytes and neutrophils and T cells. Multiple cell types are likely to contribute. The liver is also likely to be conditioned toward the default state of tolerance. This is because it sees a high level of foreign products from the large and small intestine, as the liver gets most of its blood from the hepatic portal vein, which first passes through the gastrointestinal tract.

Thus, antigens derived from apoptotic cells are presented to the immune system by cells that phagocytose them, resulting in antigen-specific tolerance (Getts et al., 2013).

### Red Blood Cells (RBC, Erythrocyte) are One Major Cell Type Removed on a Regular Basis

The number of apoptotic cells that are cleared each day is enormous. Many of these are red blood cells (RBCs), which are replaced at the rate of roughly 200 billion per day (Nagata et al., 2010). All other cell death occurring on a regular basis amounts to ˜1-2% of this amount. RBCs are cleared in the bone marrow, the liver and the spleen, where phagocytes such as macrophages and DC process and present MHC-associated antigens to T cells.

RBCs can act as antigen carriers of antigens to which tolerance is desired. Loading of RBCs with antigens and delivery IV can result in tolerance to otherwise highly immunogenic proteins, such as ovalbumin (Cremel et al., 2013). Proteins to which tolerance is desired have also been coupled (in mice, not humans) to an erythrocyte-binding peptide or an erythrocyte binding antibody (Kontos et al., 2013). IV injection of these proteins resulted in tolerance induction.

Of note, the RBC binding domains used by Kontos et al are mouse-specific, reflecting the fact that RBC cell surface proteins diverge rapidly between species. Importantly, the RBC protein targeted in their study is only present on mouse RBCs. In addition, in their approach they target to all RBCs.

Apoptotic cells are generated continuously, and taken up by phagocytic cells through PS-dependent mechanisms. Roughly 200 billion cells undergo apoptotic cell death in the human body each day. The recognition and uptake of the bulk of these apoptotic cells occurs within tissues such as the liver and spleen, which dispose of senescent red blood cells and blood leukocytes (RBCs make up the bulk of the 200 billion, with leukocytes accounting for another 1%). An important mechanism for recognition of apoptotic cells in all animals involves the membrane lipid phosphatidylserine (PS). PS is normally found primarily on the intracellular leaflet of the plasma membrane, but becomes exposed on the cell surface when cells undergo apoptosis. There are a number of PS binding proteins (PSBPs) that bind PS through their PS-binding domains when it is exposed on the surface of dying cells (listed below). Some of these are found on phagocytic cells. They essentially act like receptors binding a ligand (PS), which then results in engulfment of the dead cell. Importantly, loss of some of these receptors results in autoimmune disease, suggesting that proper disposal of apoptotic cells is important for prevention of autoimmunity (Hanayama et al., 2006; Nagata et al., 2010).

Association of PS with an antigen(s) can confer immunological tolerance against that antigen. For example, it has been shown that formation of complexes between blood clotting factors such as factor FVIII and PS resulted in a reduced antibody response to FVIII in mice (Purohit et al., 2005; Ramani et al., 2008) (Gaitonde et al., 2011). This was shown to be tolerogenic in that it could be adoptively transferred to naive mice (Gaitonde et al., 2013). Thus, PS itself can be used to link FVIII in a toleragenic manner. It may be creating something that resembles a dying cell, in that the antigen has associated with it a lot of PS, which may promote uptake of the particle as though it was a dying cell.

Phosphatidylserine (PS) is a universal marker of cells undergoing apoptosis in all animals. Phosphatidylserine (PS) is exposed on the surface of the RBC and other cells undergoing apoptosis, and is a critical signal for their uptake (Lee et al., 2011). Thus, by using molecules that target PS, one can target antigens specifically to dying cells e.g., one can target the antigen to the dying cells exclusively. Also, various embodiments provided herein can be used across species.

Thus, various embodiments provided herein take advantage of the fact that antigens present on or in cells undergoing apoptosis induce tolerance. In addition, a PS-binding domain can be used to piggyback an antigen of interest onto these cells, resulting in tolerance being induced to these antigens in the same way that tolerance is induced to the many thousands of normal cellular proteins expressed by the cells undergoing apoptosis. Thus, in some embodiments, the tolerance-inducing molecule comprises an antigen against which tolerance is desired combined with a PS-binding domain from a PSBP. This combination of antigen and PSBP can be interchangeably referred to as antigen-phosophatidylserine-binding protein fusion (“APBP”) and/or an antigen-phosophatidylserine-binding protein complex (“APBC”). In such embodiments, the PSBD does the work of finding PS-presenting cells (cells that have exposed PS on the extracellular leaflet of the plasma membrane) in the body, allowing them to carry the antigen to the cells that will present the antigen in a toleragenic manner. Thus, a therapeutic molecule, such as an antibody or an enzyme, can be modified into a tolerance-inducing molecule by linking it to the PS-binding domain of a PSBP.

Thus, in some embodiments, a tolerance-inducing molecule is provided herein which comprises an antigen against which tolerance is desired combined with a molecule to target the antigen to PS on cells undergoing apoptosis. In some embodiments, tolerance can be induced to any antigen and in any species in which PS is exposed on the surface of cells undergoing apoptosis and is used as to clear the dying cell and induce tolerance.

In some embodiments, the antigen can be a therapeutic molecule. The therapeutic molecule can be a protein. In some embodiments, the protein comprises a therapeutic antibody, a therapeutic enzyme, a blood coagulation factor, a therapeutic cofactor, an allergen, proteins deficient by genetic disease, proteins with non-human glycosylation, proteins with a glycosylation pattern not present in the relevant species. In some embodiments, non-human proteins include, adenosine deaminase, pancreatic lipase, pancreatic amylase, lactase, botulinum toxin type A, botulinum toxin type B, collagenase, hyaluronidase, papain, L-Asparaginase, rasburicase, lepirudin, streptokinase, anistreplase (anisoylated plasminogen streptokinase activator complex), antithymocyte globulin, crotalidae polyvalent immune Fab, digoxin immune serum Fab, L-arginase, and L-methionase. In some embodiments the protein comprises a fully or partially synthetic proteins not normally found in the species of interest, human food antigens, human transplantation antigens, human autoimmune antigens, and/or antigens to which an immune response is initiated in autoimmune disease. In some embodiments, these include the following: proinsulin (diabetes), collagens (rheumatoid arthritis), myelin basic protein (multiple sclerosis). There are many proteins that are human autoimmune proteins, a term referring to various autoimmune diseases wherein the protein or proteins causing the disease are known or can be established by routine testing. Embodiments include testing a patient to identify an autoimmune protein and creating an antigen for use in a molecular fusion and creating immunotolerance to the protein. Embodiments include an antigen, or choosing an antigen from, one or more of the following proteins. In type 1 diabetes mellitus, several main antigens have been identified: insulin, proinsulin, preproinsulin, glutamic acid decarboxylase-65 (GAD-65), GAD-67, insulinoma-associated protein 2 (IA-2), and insulinoma-associated protein 2.beta. (IA-2.beta.); other antigens include ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S100beta, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor. In Addison's disease, main antigens include 21 hydroxylase, 17.alpha.-hydroxylase, and P450 side chain cleavage enzyme (P450scc); other antigens include ACTH receptor, P450c21 and P450c17. In premature ovarian failure, main antigens include FSH receptor and .alpha.-enolase. In autoimmune hypophysitis, or pituitary autoimmune disease, main antigens include pituitary gland-specific protein factor (PGSF) 1a and 2; another antigen is type 2 iodothyronine deiodinase. In multiple sclerosis, main antigens include myelin basic protein, myelin oligodendrocyte glycoprotein and proteolipid protein. In rheumatoid arthritis, a main antigen is collagen II. In immunogastritis, a main antigen is H.sup.+, K.sup.+-ATPase. In pernicious angemis, a main antigen is intrinsic factor. In celiac disease, main antigens are tissue transglutaminase and gliadin. In vitiligo, a main antigen is tyrosinase, and tyrosinase related protein 1 and 2. In myasthenia gravis, a main antigen is acetylcholine receptor. In pemphigus vulgaris and variants, main antigens are desmoglein 3, 1 and 4; other antigens include pemphaxin, desmocollins, plakoglobin, perplakin, desmoplakins, and acetylcholine receptor. In bullous pemphigoid, main antigens include BP180 and BP230; other antigens include plectin and laminin 5. In dermatitis herpetiformis Duhring, main antigens include endomysium and tissue transglutaminase. In epidermolysis bullosa acquisita, a main antigen is collagen VII. In systemic sclerosis, main antigens include matrix metalloproteinase 1 and 3, the collagen-specific molecular chaperone heat-shock protein 47, fibrillin-1, and PDGF receptor; other antigens include Scl-70, U RNP, Th/To, Ku, Jo1, NAG-2, centromere proteins, topoisomerase I, nucleolar proteins, RNA polymerase I, II and III, PM-Slc, fibrillarin, and B23. In mixed connective tissue disease, a main antigen is U1snRNP. In Sjogren's syndrome, the main antigens are nuclear antigens SS-A and SS-B; other antigens include fodrin, poly(ADP-ribose) polymerase and topoisomerase. In systemic lupus erythematosus, main antigens include nuclear proteins including SS-A, high mobility group box 1 (HMGB1), nucleosomes, histone proteins and double-stranded DNA. In Goodpasture's syndrome, main antigens include glomerular basement membrane proteins including collagen IV. In rheumatic heart disease, a main antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega. Any one or more of the above can be used in any of the compositions and/or methods provided herein. In some embodiments, the antigen can be any one or more of: a therapeutic antibody, a therapeutic enzyme, a blood coagulation factor, a therapeutic cofactor, an allergen, a protein deficient by genetic disease, a protein with non-human glycosylation, a non-native protein, a protein having a glycosylation pattern not present in a species, a non-human protein, a non-native protein, a synthetic protein, a recombinant protein, a human food protein allergen, including those found shrimp, shellfish, scaly fish or crustaceans, or peanut, tree nut, milk, egg, wheat, or soy, non-food protein allergens, including those found in plants and non-food animals, a human transplantation antigen, a human autoimmune antigen, an antigen to which an immune response is initiated in autoimmune disease insulin, proinsulin, preproinsulin, glutamic acid decarboxylase-65 (GAD-65), GAD-67, insulinoma-associated protein 2 (IA-2), insulinoma-associated protein 2beta (IA-213), ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S100beta, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5; b) thyroglobulin (TG), thyroid peroxidase (TPO), thyrotropin receptor (TSHR), sodium iodine symporter (NIS) and megalin; c) thyroglobulin (TG), thyroid peroxidase (TPO), thyrotropin receptor (TSHR), sodium iodine symporter (NIS), megalin, and insulin-like growth factor 1 receptor; d) calcium sensitive receptor; e) 21-hydroxylase, 17.alpha.-hydroxylase, P450 side chain cleavage enzyme (P450scc), ACTH receptor, P450c21 and P450c17; f) FSH receptor and .alpha. enolase; g) pituitary gland-specific protein factor (PGSF) 1a, PGSF 2, and type 2 iodothyronine deiodinase; h) myelin basic protein, myelin oligodendrocyte glycoprotein and proteolipid protein; i) collagen II; j) H⁺,K⁺-ATPase; k) intrinsic factor; l) tissue transglutaminase and gliadin; m) tyrosinase, and tyrosinase related protein 1 and 2; n) acetylcholine receptor; o) desmoglein 3, desmoglein 1, desmoglein 4, pemphaxin, desmocollins, plakoglobin, perplakin, desmoplakins, and acetylcholine receptor; p) BP180, BP230, plectin and laminin 5; q) endomysium and tissue transglutaminase; r) collagen VII; s) matrix metalloproteinase 1 and 3, the collagenspecific molecular chaperone heat-shock protein 47, fibrillin-1, PDGF receptor, Scl-70, U1 RNP, Th/To, Ku, Jol, NAG-2, centromere proteins, topoisomerase I, nucleolar proteins, RNA polymerase I, II and III, PM-Slc, fibrillarin, and B23; t) U1 snRNP; u) SS-A, SS-B, fodrin, poly(ADP-ribose) polymerase, and topoisomerase v) SS-A, high mobility group box 1 (HMGB1), nucleosomes, histone proteins and double-stranded DNA; w) glomerular basement membrane proteins including collagen IV; x) cardiac myosin; and y) aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega; z) antithrombin-III, protein C, factor VIII, factor IX, growth hormone, somatotropin, insulin, pramlintide acetate, mecasermin (IGF-1), beta-gluco cerebrosidase, alglucosidase-alpha, laronidase (alpha Liduronidase), idursuphase (iduronate-2-sulphatase), galsulphase, agalsidase-beta (alpha-galactosidase), alpha-1 proteinase inhibitor, and albumin; aa) adenosine deaminase, pancreatic lipase, pancreatic amylase, lactase, botulinum toxin type A, botulinum toxin type B, collagenase, hyaluronidase, papain, L-Asparaginase, uricase, lepirudin, streptokinase, anistreplase (anisoylated plasminogen streptokinase activator complex), antithymocyte globulin, crotalidae polyvalent immune Fab, digoxin immune serum Fab, L-arginase, and L methionase; bb) conarachin (Ara h 1), allergen II (Ara h 2), arachis agglutinin, conglutin (Ara h 6), 31 kda major allergen/disease resistance protein homolog (Mal d 2), lipid transfer protein precursor (Mal d 3), major allergen Mal d 1.03D (Mal d 1), alpha lactalbumin (ALA), lactotransferrin, actinidin (Act c 1, Act d 1), phytocystatin, thaumatin-like protein (Act d 2), kiwellin (Act d 5), 2S albumin (Sin a 1), 11S globulin (Sin a 2), lipid transfer protein (Sin a 3), profilin (Sin a 4), profilin (Api g 4), high molecular weight glycoprotein (Api g 5), Pen a 1 allergen (Pen a 1), allergen Pen m 2 (Pen m 2), tropomyosin fast isoform, high molecular weight glutenin, low molecular weight glutenin, alpha- and gamma-gliadin, hordein, secalin, avenin, major strawberry allergy Fra a 1-E (Fra a 1), and profilin (Mus xp 1); and/or cc) subunits of MHC class I and MHC class II haplotype proteins, and single-amino-acid polymorphisms on minor blood group antigens including RhCE, Kell, Kidd, Duffy and Ss.

In some embodiments, the protein can be a therapeutic antibody, a therapeutic enzyme or a therapeutic protein, which is used for a therapeutic purpose but against which an immune response should be prevented. For example, in some embodiments, the protein can be a blood coagulation factor with a therapeutic benefit in a patient with a defective blood clotting pathway. In some embodiments, the therapeutic protein can be a therapeutic cofactor for an enzymatic reaction in a patient with, for example, a metabolic defect. A metabolic defect can be caused due to acid-base imbalance, metabolic brain diseases, calcium metabolism disorders, DNA repair-deficiency disorders, glucose metabolism disorders, hyperlactatemia, iron metabolism disorders, lipid metabolism disorders, Malabsorption syndromes, metabolic syndrome X, inborn error of metabolism, mitochondrial diseases, phosphorus metabolism disorders, porphyrias, proteostasis deficiencies, metabolic skin diseases, wasting syndrome, water-electrolyte imbalance or a combination thereof. In some embodiments, the protein is an allergen, for example a pollen protein, a food allergen, or a protein produced by some other animal or plant that results in an allergic response. In some embodiments, the protein can be a protein that is deficient in the host due to a genetic disease which results in the protein not being produced (or produced adequately and/or properly). In some embodiments, the protein can be a human food antigen to which an individual is allergic, for example a shrimp protein or a protein found in shellfish, scaly fish or crustacean, or peanut, tree nut, milk, egg, wheat, or soy. Examples of specific proteins and the organisms they derive from include peanut: conarachin (Ara h 1), allergen II (Ara h 2), arachis agglutinin, conglutin (Ara h 6); from apple: 31 kda major allergen/disease resistance protein homolog (Mal d 2), lipid transfer protein precursor (Mal d 3), major allergen Mal d 1.03D (Mal d 1): from milk: .alpha. lactalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen m 2), tropomyosin fast isoform; from wheat and/or other cerials: high molecular weight glutenin, low molecular weight glutenin, alpha- and gamma gliadin, hordein, secalin, avenin; from strawberry: major strawberry allergy Fra a 1-E (Fra a 1), from banana: profilin (Mus xp 1).

In some embodiments, the protein can be a human autoimmune antigen (antigens to which an immune response is initiated in autoimmune disease) against which neither central nor peripheral immune tolerance developed. In some embodiments, the protein can be a human transplantation antigen, for example, an antigen of a transplanted organ such that the organ is not rejected in the organ recipient. In some embodiments, the protein has a non-human glycosylation pattern or, more generally, the protein has a glycosylation pattern that is not present in the species of interest. In some embodiments, the protein is a non-human protein or, more generally, does not belong to the species of interest or is a synthetic protein not normally found in the species of interest.

In some embodiments, a general method for associating proteins (such as protein antigens) of interest to a dying cell in any vertebrate for the purposes of inducing tolerance is provided. Fluorescently tagged or otherwise labeled versions of proteins containing PS-binding domains have been used to visualize dying cells, taking advantage of the fact that the PS-binding domain recruits these proteins to dying cells (reviewed in (Kim et al., 2015; Neves and Brindle, 2014; Zeng et al., 2015)). PS-binding domains have also been used to recruit other cell death-inducing proteins to tumors, which have a large fraction of dying cells (e.g. (Guillen et al., 2015; Qiu et al., 2013)). In addition, antigens have been loaded into dying cells to induce tolerance (discussed above). However, PS-binding domains have not been used to link antigens to dying cells for the purposes of inducing tolerance against those antigens.

In some embodiments, the PSBP is covalently linked to the therapeutic molecule to form the APBP/APBC. In some embodiments, the PSBD can be chemically (covalently) coupled to the antigen of interest to bring about linkage between the antigen and the PSBD. In some embodiments, the PSBP is directly covalently linked to the antigen. In some embodiments, the PSBP is indirectly covalently linked to the antigen. In some embodiments, the indirect linkage of the PSBP to the antigen is via a linker. In some embodiments, the linker is a chemical linker. In some embodiments, the linker is a peptide linker. In some embodiments, the PSBD can be coupled chemically (covalently) to the antigen of interest to bring about linkage between the antigen and the PSBD (such as, a disulfide bond, for example). In some embodiments, the PSBD can be associated non-covalently with the antigen of interest, examples include, but are not limited to, charge-charge interactions, association in lipid complexes, antibody or other protein mediated binding, or other nanoparticles. Some exemplary embodiments of PS-binding peptides or proteins, or protein domains include, but are not limited to, Tim1-4 proteins: (Kobayashi et al., 2007; Miyanishi et al., 2007; Santiago et al., 2007; Schweigert et al., 2014; Tietjen et al., 2014) Lactadherin/MFG-E8: (Dasgupta et al., 2008; Hanayama et al., 2002; Reddy Nanga et al., 2007; Shao et al., 2008; Ye et al., 2013) Stabilin-1 and Stabilin-2: (Park et al., 2008a) (Park et al., 2008b) Gas6/protein S: (Anderson et al., 2003; Ishimoto et al., 2000; Morizono et al., 2011) C300a: (Nakahashi-Oda et al., 2012; Simhadri et al., 2012) BAI1: (Park et al., 2007) RAGE: (He et al., 2011) PDK1: (Lucas and Cho, 2011) Annexins: (Rosenbaum et al., 2011) C1Q: (Paidassi et al., 2008) Factor V in thrombin cascade: (Srivastava et al., 2001) Drosophila Draper: (Tung et al., 2013) Stapylococcal SSL10: (Itoh et al., 2012) PSR-1: (Yang et al., 2015); Various peptides such as CLSYYPSYC (SEQ ID NO: 22) (Thapa et al., 2008)(Kim et al., 2015), AREDGYDGAMDY (SEQ ID NO: 7) (Igarashi et al., 1995), LIKKPF (SEQ ID NO: 8), CLIKKPF (SEQ ID NO: 9), PGDLSR (SEQ ID NO: 10), CPGDLSR (SEQ ID NO: 11) (Burtea et al., 2009), FNFRLKAGQKIRFG (SEQ ID NO: 12) (Igarashi et al., 1995b), FNFRLKAGAKIRFG (SEQ ID NO: 13), FNFRLKVGAKIRFG (SEQ ID NO: 14), FNFRLKTGAKIRFG (SEQ ID NO: 15), FNFRLKCGAKIRFG (SEQ ID NO: 16) (Xiong et al., 2011), RSRRMTRRARAA (SEQ ID NO: 17) (Nakai et al., 2005), TLVSSL (SEQ ID NO: 18) (Laumonier et al., 2006), TRYLRIHPRSWVHQIALRLRYLRIHPRSWVHQIALRS (SEQ ID NO: 19), TRYLRLHPRSWVHQLALRLRYLRLHPRSWVHQLALRS (SEQ ID NO: 20) (Kuriyama et al., 2009), KKKKRFSFKKSFKLSGFSFKKNKK (SEQ ID NO: 21) (Kim et al., 2015; Morton et al., 2013), saposin C (Qi and Grabowski, 2001), and phosphatidylserine-binding monoclonal antibodies (e.g. (Gong et al., 2013)).

In some embodiments, other related proteins and peptides that bind PS can be identified and used by those with skill in the art through binding assays to various lipids, homology searches to known PS-binding domains, and through isolation and sequencing of proteins or peptides from PS-containing membranes.

### Compositions

In some embodiments, the antigen can be supplied associated with the PSBP (that is, as part of a tolerance inducing molecule), in combination with an amount of free antigen (or antigen that is not associated with the PSBP, for example, a free therapeutic molecule). In some embodiments, a composition is provided which is a mixture of any one of the therapeutic molecules described herein in the form of a tolerance-inducing molecule and a free therapeutic molecule, wherein the free therapeutic molecule is not present as a tolerance-inducing molecule. In some embodiments, the free therapeutic molecule is introduced simultaneously with the tolerance-inducing molecule. In some embodiments, tolerance can be desired against a therapeutic molecule, such as an antibody or enzyme, but it can additionally be desired to introduce the therapeutic antibody or enzyme to specifically manipulate a physiological and/or biochemical process in some way. For example, it can be desired to induce immunological tolerance against the therapeutic molecule first before introducing the therapeutic molecule to manipulate the physiological and/or biochemical process Thus, in some embodiments, the therapeutic molecule is introduced as a tolerance-inducing molecule as well as a free therapeutic molecule separately (not associated with the PSBP). In some embodiments, the free therapeutic molecule is introduced in isolation e.g., separately from the tolerance-inducing molecule. In some embodiments, the free therapeutic molecule is introduced subsequent to the introduction of the tolerance-inducing molecule. In some embodiments the PSBP-fusion protein is introduced alone, and functions both to induce tolerance and to perform the therapeutic function.

Thus, in some embodiments, a composition comprising a mixture of any one of the tolerance-inducing molecules described herein and a free therapeutic molecule is provided. The free therapeutic molecule is not associated with the antigen, and the antigen of the tolerance-inducing molecule and the free therapeutic molecule are both at least one of: a therapeutic antibody, a therapeutic enzyme, a blood coagulation factor, a therapeutic cofactor, an allergen, a protein deficient by genetic disease, a protein with non-human glycosylation, a non-native protein, a protein having a glycosylation pattern not present in a species, a non-human protein, a non-native protein, a synthetic protein, a recombinant protein, a human food allergen, a non-food allergen derived from a plant or animal, a human transplantation antigen, a human autoimmune antigen, an antigen to which an immune response is initiated in autoimmune disease, and/or insulin.

In some embodiments of the composition, the tolerance-inducing molecule is present in a first amount and the free therapeutic molecule is present in a second amount. In some embodiments of the composition, the first amount is less than the second amount. In some embodiments of the composition, the first amount is about the same as the second amount. In some embodiments of the composition, the first amount is more than the second amount. The first and second amounts can vary depending on the condition and/or situation and/or the therapeutic molecule. The effective first and second amounts for a condition and/or situation and/or therapeutic molecule can be determined either empirically or based on an educated guess by one skilled in the art. The first amount can range from about 1 ng/ml to 1 mg/ml and the second amount can range from about 1 ng/ml to 1 mg/ml.

In some embodiments of the composition, the antigen of the tolerance-inducing molecule is the same type of molecule as the free therapeutic molecule. In some embodiments of the composition, the antigen of the tolerance-inducing molecule and the free therapeutic molecule are both therapeutic molecules. In some embodiments of the composition, the antigen of the tolerance-inducing molecule and the free therapeutic molecule are both proteins. In some embodiments of the composition, when the tolerance-inducing molecule and the free therapeutic molecule are both proteins, the antigen of the tolerance-inducing molecule and the free therapeutic molecule are about 70% to about 100% identical. In some embodiments of the composition, the antigen of the tolerance-inducing molecule and the free therapeutic molecule are about 70, 75, 80, 85, 90, 95 or 100% identical.

In some embodiments, a composition comprising both the tolerance-inducing molecule and the free therapeutic molecule is different from a composition which is used in a situation where it is only desired to dampen or eliminate an ongoing immune response to an endogenous protein. In some embodiments, the protein antigen can only be introduced as a tolerance-inducing molecule. Thus, in some embodiments, the antigen is introduced only as an APBC (without any free form of the antigen being supplied).

In some embodiments, the approaches provided herein can be used as a vectored approach, providing for long term tolerance maintenance; however, in other approaches (noted above) the antigen is be coupled with a PSBP and then injected into the individual each time tolerance is to be induced. In some embodiments, vectored approaches (detailed further below), in which the individual expresses a tolerance inducing and/or maintaining fusion protein for prolonged periods of time following introduction of DNA into somatic cells such as skeletal muscle, provide another approach. In some embodiments, compositions and methods for bringing about immunological tolerance to specific proteins, peptides or other molecules are provided herein. In some embodiments, vector based approaches for bringing about antigen-specific tolerance are provided. In some embodiments, passive infusion is used.

In some embodiments, a recombinant genetic construct is provided. In some embodiments, the construct comprises a vector that comprises a nucleic acid sequence encoding any one or more of the therapeutic molecules described herein against which tolerance is desired. In some embodiments, the vector is a viral vector. In some embodiments, the vector is a lentiviral vector. In some embodiments, the vector is an adeno-associated viral (AAV) vector. In some embodiments the vector is a plasmid, a minicircle, a closed linear DNA, a doggybone DNA, dumbbell DNA, or other form of double-stranded DNA. In some embodiments the vector is linear or circular RNA; it may contain only the naturally-occurring nucleotides, or it may contain modified nucleotides. The genetic construct can comprise a gene that encodes a protein or peptide to which tolerance is desired, designed to be expressed as a protein fusion with the PSBD from any of the proteins with a PSBD listed above, the peptides listed above, or PSBDs generated by individuals with skill in the art. The genetic construct can be configured to be delivered and expressed in an animal and/or a subject.

In some embodiments, a method is provided for the induction of antigen specific tolerance. The method can involve AAV-mediated delivery of an antigen linked through genetic fusion with a PSBD. In some embodiments, any one of a number of different gene delivery methods can be used. These include but are not limited to liposomes, nanoparticles, virus-like particles, phage, or complexes with cell penetrating peptides. In some embodiments, a combination of one or more of these gene delivery methods can be used in combination with one or more DNA expression construct, which can be a virus, a plasmid, a minicircle, a closed linear DNA, a doggybone DNA, or other form of double-stranded DNA. RNA vectors may also be utilized.

In some embodiments, a composition comprising an associated virus (AAV) vector having an AAV capsid having packaged therein nucleic acid sequences comprising an AAV 5′ inverted terminal repeat (ITR), a sequence encoding a polypeptide which encodes a protein or peptide for which tolerance is provided. It can be linked to a PSBD, under control of regulatory sequences which direct expression of a PSBD-linked fusion protein, and an AAV 3′ ITR. The polypeptide can be a tolerance-inducing antigen-PSBD fusion protein (referred to as an antigen-PSBD), and this can be linked as a fusion protein to other protein domains that extend the half-life of the tolerance-inducing fusion protein, such as an immunoglobulin Fe domain, or albumin, or an albumin-binding domain. In some embodiments, the half-life and level of expression of the tolerance-inducing fusion protein can be altered using other protein domains or a protein destabilizing domain that can be used to interfere with protein conformation.

In some embodiments, the viral composition can contain about 10⁸ to about 5×10¹⁴ vector particles per 1 mL aqueous suspension. In some embodiments, the viral composition can comprise about 10⁸, 10⁹, 10¹⁰, 10¹¹, 10¹², 10¹³, 1×10¹⁴, 2×10¹⁴, 3×10¹⁴, 4×10¹⁴ or 5×10¹⁴ vector particles per mL aqueous solution, including any range between any two of the preceding values. In some embodiments, the composition is adapted for delivery to non-human animals. In some embodiments, the composition is adapted for delivery to humans. In some embodiments, the composition is formulated for intramuscular delivery. In some embodiments, the composition is formulation for intravenous delivery. In some embodiments, a lyophilized composition comprising the AAV expressed antigen-PSBD fusion protein is provided. In some embodiments, a reconstituted composition comprising the lyophilized composition and about 10⁹ to about 5×10¹³ vector particles per 1 mL aqueous suspension is provided. In some embodiments, a reconstituted composition comprising the lyophilized composition and about 10⁹, 10¹⁰, 10¹¹, 10¹², 1×10¹³, 2×10¹³, 3×10¹³, 4×10¹³ or 5×10¹³ vector particles per mL aqueous solution is provided. In some embodiments, the non-viral DNA or RNA composition can comprise about 10⁸, 10⁹, 10¹⁰, 10¹¹, 10¹², 10¹³, 1×10¹⁴, 2×10¹⁴, 3×10¹⁴, 4×10¹⁴ or 5×10¹⁴ molecules (with molecule meaning one copy of a vector sufficient to bring about expression of the therapeutic) per mL aqueous solution, including any range between any two of the preceding values.

In some embodiments, a pharmaceutical composition is provided. It can comprise the genetic construct described herein and a pharmaceutically acceptable carrier. The concentration of the genetic construct can be about 0.1 to about 1 mg/ml. In some embodiments, the concentration of the genetic construct can be about 0.1. 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 50, 100, 200, 300, 400, 500, 600, 700, 800, 900, or 1000 ng/ml, including any range between any two of the preceding values. In some embodiments, the pharmaceutical composition can comprise antigen-PSBP protein preparation. In some embodiments, the pharmaceutical composition can be a combination of an antigen-PSBP preparation and the genetic construct that expresses the antigen-PSBP.

In some embodiments, the pharmaceutically acceptable carrier can be combined with a nucleic acid-based genetic construct of the tolerance-inducing molecule. In some embodiments, the pharmaceutically acceptable carrier can be combined with a protein preparation of the tolerance-inducing molecule. In some embodiments, the pharmaceutically acceptable carrier can be combined with a combination of the protein preparation and genetic construct of the tolerance-inducing molecule. In some embodiments, the pharmaceutically acceptable carrier can be a liquid or aqueous carrier. In some embodiments, oral delivery can be performed by using a pharmaceutically acceptable carrier that is capable of withstanding degradation by digestive enzymes in the gut of an animal. Non-limiting examples of such carriers include plastic capsules or tablets, such as those known in the art. In some embodiments, topical delivery can be achieved with a lipophilic reagent (e.g., DMSO) that is capable of passing through and into the skin. In some embodiments, nasal delivery can be performed by using a pharmaceutically acceptable carrier.

In some embodiments, a method of inducing immunological tolerance is provided. The method can involve administering a composition provided herein to a subject in which immunological tolerance is to be induced. The composition can be selected from any of the compositions described herein. For example, the composition can comprise antigens coupled through covalent or non-covalent options to a PSBD can be delivered to an individual through IV injection, nasal or oral delivery, or through any of the routes noted: parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, and/or transdermal.

In some embodiments, a method of tolerance induction in an animal and/or human is provided. In some embodiments, the method comprises administration of a tolerogenic composition to an animal and/or a subject, wherein the tolerogenic composition comprises either a genetic construct comprising a nucleic acid sequence expressing any of the APBP or an APBC as a protein preparation or a combination of both a genetic construct and protein preparation. In some embodiments, the method comprises the use of a tolerogenic composition that can comprise the genetic construct described herein comprising any of the APBPs and/or APBCs as a preparation that can be administered to an animal and/or a subject. In some embodiments, the method comprises the use of a tolerogenic composition that is configured to be delivered to an animal through intramuscular injection, subcutaneous injection, intravenous injection, intraperitoneal injection, oral delivery, electroporation through the skin, sonication, and/or nasal inhalation. The method can comprise administering to the animal or human a genetic construct described above or herein.

In some embodiments, a method for inducing antigen-specific tolerance is provided, which comprises delivering an effective amount of the composition or purified rAAV described herein. The effective amount required can be determined empirically by one skilled in the art, given the present disclosure. The method can involve expressing the antigen-PSBD fusion under the control of a constitutive promoter or a regulatable promoter. In some embodiments, the promoter is an inducible promoter. In some embodiments, the promoter is induced by a small molecule drug. In some embodiments, the promoter is constitutive, and the antigen-PSBD (phosphatidylserine binding domain) fusion expression can be inhibited through expression in the transfected cells of small RNAs that inhibit expression of the fusion protein, or a recombinase that separates the enhancer-promoter sequences from the antagonist coding sequence, or some portion thereof. In some embodiments, expression of the fusion protein, which can also be inducible, can be silenced through removal of the transgene-expressing cells, as a result of inducible activation of a co-expressed suicide gene (Jones et al., 2014).

In some embodiments, tolerance can be induced to a wide variety of antigens, particularly proteins, by linking them to a PS-binding domain from a PSBP, followed by introduction of these reagents into, for example, the circulatory system of the individual/subject. For example, the subject has or is at risk of at least one of the following: Factor VIII deficiency, an autoimmune disease, type 1 diabetes, multiple sclerosis, lupus, rheumatoid arthritis; a transplant related disorder, graft vs. host disease (GVHD), allergic reaction; immune rejection of biologic medicines including: monoclonal antibodies, replacement proteins including FVIII and/or insulin, a therapeutic toxin, including Botulinum toxin; and the management of immune response to infectious disease. In some embodiments, the antigens to which tolerance is to be induced can be derived from other organisms. For example, in some embodiments, the antigen can be from companion animals such as dogs and cats.

In some embodiments, introduction can involve passive infusion of the composition and/or tolerance-inducing molecule through an IV injection. It can also involve vectored expression of a chimeric protein using many different kinds of vectors. Finally, in some contexts it is possible to use oral or suppository delivery to provide antigens in a PS-binding form that promotes tolerance once the chimera passes into the body. An example can involve genetic fusion of an antigen of interest to an immunoglobulin Fc domain, along with a PS-binding domain. Examples of other delivery sites include intramuscular or intravenous injection, resulting in expression in tissues such as skeletal muscle, liver, brain and kidney. Examples using nasal or oral delivery include expression in the respiratory and digestive systems, respectively. In some embodiments, delivery is by one or more of the following: parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal. In some embodiments, the composition to be delivered can be configured for delivery via one or more of the above noted routes.

In some embodiments, one or more of the compositions (including any tolerance-inducing molecule) can be applied in any number of conditions. These include autoimmune disease such as type 1 diabetes, Multiple Sclerosis, Lupus, and Rheumatoid Arthritis; Transplant related disorders such as Graft vs. Host disease (GVHD); Allergic reactions; Immune rejection of biologic medicines such as monoclonal antibodies, replacement proteins such as FVIII or Insulin, the use of therapeutic toxins such as Botulinum toxin; and the management of immune response to infectious disease whether acute or chronic. Other examples include inappropriate immune response to a therapeutic antibody, a therapeutic enzyme, a blood coagulation factor, a therapeutic cofactor, an allergen, a protein deficient by genetic disease, a protein with non-human glycosylation, a non-native protein, a protein having a glycosylation pattern not present in a species, a non-human protein, a non-native protein, a synthetic protein, a recombinant protein, a human food allergen, a non-food allergen derived from a plant or animal, a human transplantation antigen, a human autoimmune antigen, an antigen to which an immune response is initiated in autoimmune disease, and/or insulin.

Any of the methods and/or compositions provided herein in regard to humans can also be provided to one or more companion animals or animals domesticated for commercial interest. A companion animal can be, for example, dog, cat, guinea pig. An animal domesticated for commercial interest can be, for example, goat, sheep, cow, pig, and chicken.

### EXAMPLES

Detailed below are text and references to figures illustrating some examples of how antigen-specific tolerance can be implemented.

### Example 1

### Antibody-PSBD Fusion

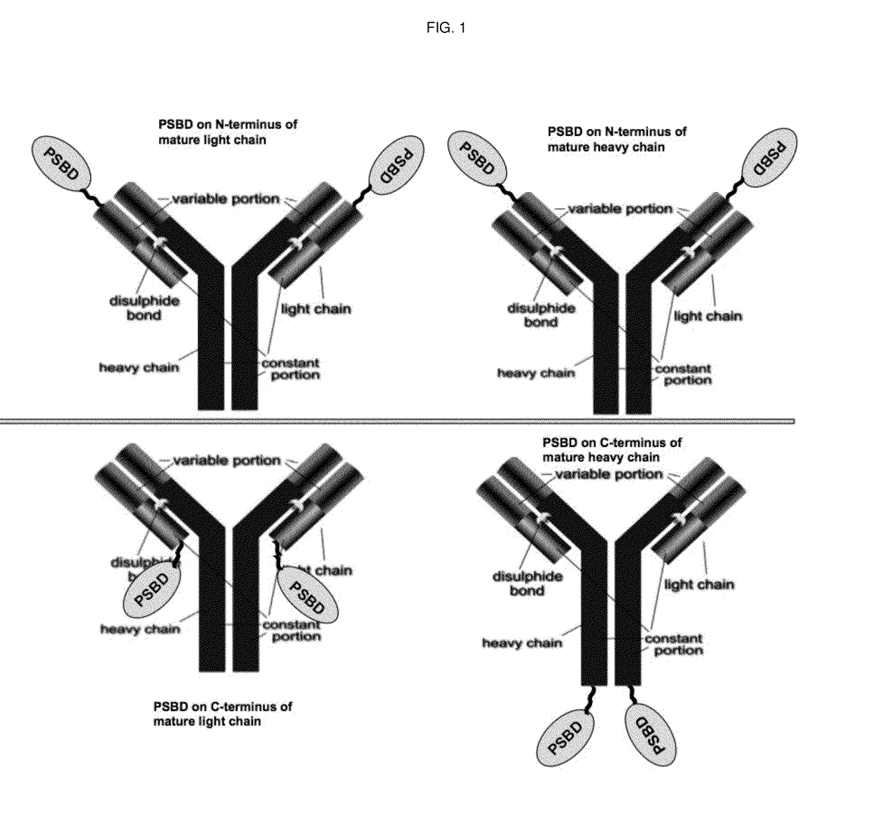

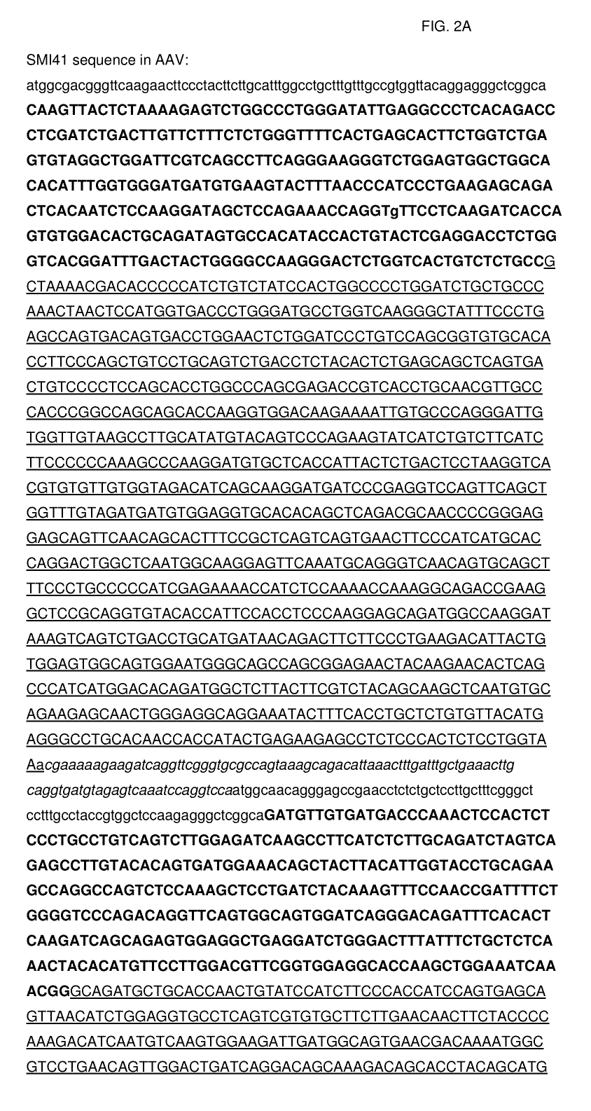

The PSBD is attached to an antibody through genetic fusion, with a flexible linker (black squiggles) separating the antibody from the PSBD as shown in some embodiments in FIG. 1. Other antigen-PSBD fusions can be generated using similar standard approaches.

### Example 2

### A Mouse AntibodyPSBD Fusion Designed to Bind GnRH

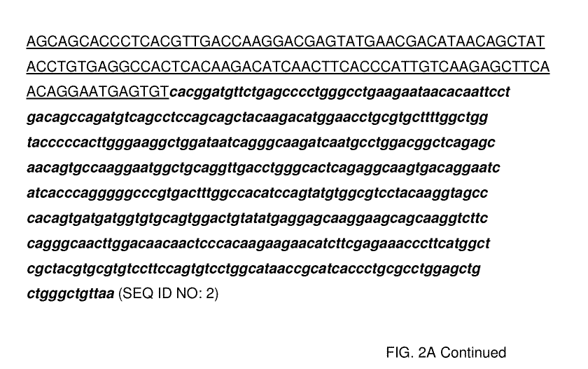

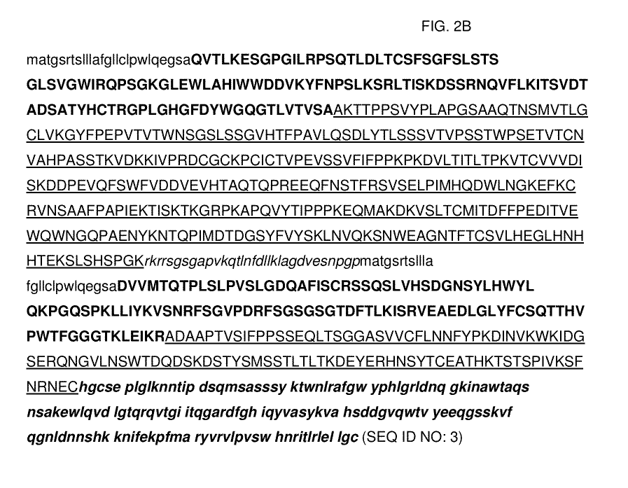

GnRH is a reproductive hormone important for fertility in mammals. The antibody is designed to be expressed from N- to C-terminus as: signal peptide-heavy chain-F2A peptide-signal sequence-light chain-PSBD (C2 domain from lactadherin) shown in a nucleotide sequence embodiment in FIG. 2A (SEQ ID NO: 2) and the translated protein sequence in FIG. 2B (SEQ ID NO: 3). In the both the nucleotide and protein sequences the signal peptide coding sequences is shown in lowercase letters. The variable regions of the antibody are shown in bold, constant regions of the antibody are underlined. The F2Aopt peptide coding sequence is in lowercase letters and italicized and the PSBD in italicized and in bold. In this configuration it is expected that some fraction of the antibody will be taken up with dying cells and presented in a toleragenic manner. In this way, the antibody, even if it is not adapted to the species of interest, will be less likely to induce an immune response that results in neutralization of antibody function.

### Example 3

### A Protocol for Generating AAV Carrying a PSBD-Fusion Protein, Suitable for Injection into Muscle is Provided Below

Genes encoding the fusion protein of interest are introduced into an AAV2/8 vector, such as that described in (Balazs, A. B., Chen, J., Hong, C. M., Rao, D. S., Yang, L., and Baltimore, D. (2012). Antibody-based protection against HIV infection by vectored immunoprophylaxis. Nature 481, 81-84), (Li et al., 2015).

To generate virus, 293T cells are seeded in 15 cm plates at 3.75×10̂6 cells per plate in 25 ml DMEM medium supplemented with 10% fetal bovine serum, 1% penicillin-streptomycin mix and 1% glutamine in a 5% CO2 incubator at 37° C. After three days culture, media is changed to 15 ml of fresh media and two hours later, the AAV backbone vector, which contains the fusion proteinencoding gene is co-transfected with helper vectors pHELP (Applied Viromics) and pAAV 2/8 SEED (University of Pennsylvania Vector Core) at a ratio of 1:4:8 using BioT transfection reagent (Bioland Scientific). AAV virus is then collected from culture supernatant at 36, 48, 72, 96 and 120 h after transfection and these fractions pooled.

Virus can be purified in several different ways. In one approach virus is purified by filtering the virus culture supernatant through a 0.2 mm filter, followed by centrifugation at 110,527 g for 1.5 h. The virus pellet is dissolved in DMEM and stored at −80° C.

In a second approach, virus is obtained from the supernatant after spinning out other cellular components. PEG solution (40% polyethylene glycol in 2.5M NaCl) is added to the supernatant at a volume ratio of 1:4, and gently mixed at 4° C. overnight to precipitate virus. Precipitated virus is pelleted at 4,000 g for 30 min and re-suspended in 10 ml MEM. To remove PEG residue and concentrate the virus, this solution is loaded onto 100 kDa MWCO centrifugal filters (Millipore) and spun at 3220 g at 4° C. until ˜1 ml retentate remained. Fresh MEM is added to the filter and this process is repeated three times. Final virus solution is about 2 ml total and stored at −80° C.

Virus prepared as above can be injected directly into skeletal muscle using standard delivery devices (syringe and needle), as with vaccines or other therapeutics.

Detailed below are some common examples of inappropriate immune activation and how one can treat them using the approaches as disclosed herein.

### Example 4

### Factor VIII, Factor IX, Factor XI Deficiency

People with mutations in Factor VIII, or in the alternative IX, or in the alternative XI, or in the alternative, other genes involved in blood clotting, lack the ability to clot blood appropriately. A current treatment is to give them recombinant versions of the protein. However, since for them this is seen as a foreign protein (they are mutants), some fraction of individuals make antibodies to the protein. This then limits the effectiveness of the treatment.

In order to overcome this, a fusion protein, or a gene that expresses a version of the protein, that carries a PS-binding domain (PSBD) can be generated. This can allow the protein to be presented in a toleragenic context.

Including the PSBD may—or may not—reduce the half-life of the protein therapeutic as it will now be targeted for phagocytosis and degradation through association with dying cells. In order to limit this, one may introduce two versions of the protein into the individual, one that is wildtype, and another that carries the PSBD. The version carrying the PSBD will be phagocytosed along with dying cells and induce tolerance to the wildtype protein. The wildtype version will have its normal half-life, and be able to carry out its normal function.

### Example 5

### Antibodies to a Monoclonal Antibody

Monoclonal antibodies are provided to humans and animals for a variety of therapies. Often the antibody has been humanized in the hope of preventing an unwanted immune response. Sometimes this fails. Humans also have amino acid sequence differences (polymorphisms) in the constant regions. There is evidence that these differences can sometimes lead to anti-antibody immune responses. In addition, sometimes antibodies are generated against the variable regions of a monoclonal antibody (Jefferis and Lefranc, 2009; Pandey and Li, 2013). Finally, in some situations one can use antibodies that are only partially humanized or similarly modified so as to be seen as self for a particular species.

Given the above, one can administer antibodies to animals or humans in a way that does not provoke an immune response. This can be done by including a PS-binding domain linked to the N- or C-terminus of the heavy or light chain of an antibody (see the various embodiments in FIG. 1).

One can infuse individuals and animals with two different genetic constructs, which can be DNA-based or RNA-based, viral or non-viral: one that just expresses the antibody alone, and a second that carries the antibody with a PS-binding domain, with the latter one perhaps making up only some fraction of the total. The PSBD-linked antibody can induce tolerance to the antibody, while the wild-type antibody goes and performs its normal function.

One can also infuse individuals directly (IV) with compositions consisting of some fraction of wildtype antibody and some fraction PSBD-linked, either covalently or non-covalently.

In addition, one can also infuse the individual with only the version of the antibody that is linked to the PSBD. The individual can also be induced to express, following gene-therapy-mediated delivery, only a version of the antibody that carries the PSBD.

### Example 6

### Autoimmune Disease

If the identity of the protein that antibodies or reactive T cells are targeting is known, one can make a version of the protein, or a vector that expresses the protein or some fraction of the protein (that includes the antigen), linked to a PSBD. This can induce tolerance to the wild-type version of the protein when infused into the person or animal.

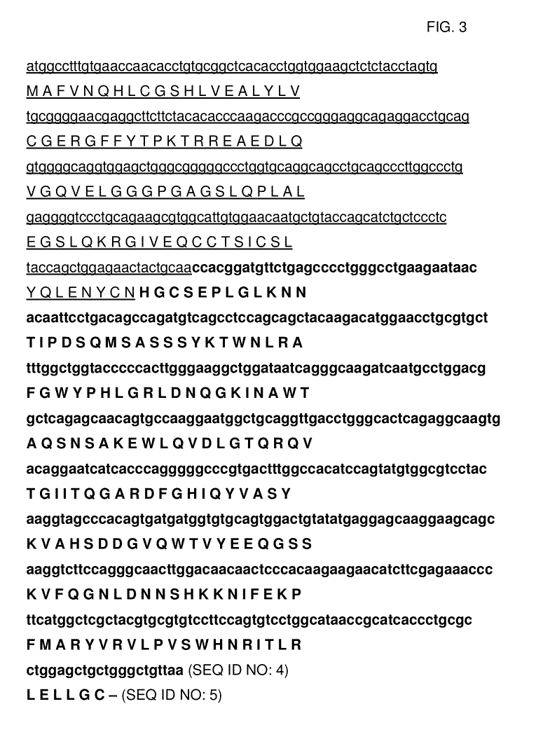

In one example, one can induce tolerance to insulin, to which antibodies are often made in Type I diabetes (a possible construct for this is outlined in FIG. 3; pre-pro-insulin sequence is underlined and the C2 domain of lactadherin is shown in bold; nucleotide sequence—SEQ ID NO: 4; protein sequence—SEQ ID NO: 5). The protein (including the antigen, and/or other sequence versions of the antigen) can be provided as a protein, injected IV, or it could be provided as a transgene in a gene delivery vector designed to synthesize a protein that comprises the protein antigen of interest fused through genetic engineering approaches to a PSBD.

This vector can be provided to the individual in the form of an adeno-associated virus (AAV) vector, injected into muscle or, in the alternative, some other tissue. The fusion protein can be synthesized by muscle, secreted, and enter the blood stream where it is able to access apoptotic cells, the bulk of which are red blood cells. The vector can also be provided to the individual in the form of a non-viral, DNA or RNA-based vector.

Examples of diseases or other conditions where antigen-specific tolerance is desired include, but are not limited to, those provided herein. Possible implementations are briefly described.

### Example 7

### Application to Allergy

Allergen-specific regulatory T cells play an important role in controlling the development of allergy and asthma. Both naturally occurring CD4/CD25 regulatory T cells and secondary TRegs (antigen-specific regulatory T cells), both expressing the transcription factor FOXp3, have been shown to inhibit the inappropriate immune responses involved in allergic diseases. A number of recent studies indicate that regulatory T cells play an important role in controlling the overdevelopment of T-helper type 2 biased immune responses in susceptible individuals, not only in animal models, but in humans as well. Recent studies indicate that T regulatory cells also suppress T cell costimulation by the secretion of TGF-beta and IL-I0, indicating an important role of T regulatory cells in the regulation of allergic disorders.

Given the above, impaired expansion of natural or adaptive regulatory T cells leads to the development of allergy, and treatment to induce allergen-specific regulatory T cells can provide curative therapies for allergy and asthma.

Where one wishes to provide for the prevention and therapy of asthma or allergy, one can cause the induction of regulatory T cells. These cells can be induced in response to presentation of antigen (that is an allergy antigen, a shrimp protein or a protein found in shellfish, scaly fish or crustacean, or peanut, tree nut, milk, egg, wheat, or soy) associated with a PSBD. The antigen can be covalently linked to the PSBD and/or non-covalently linked to the antigen.

Antigens of interest, and the organism they derive from include the following: peanut: conarachin (Ara h 1), allergen II (Ara h 2), arachis agglutinin, conglutin (Ara h 6); from apple: 31 kda major allergen/disease resistance protein homolog (Mal d 2), lipid transfer protein precursor (Mal d 3), major allergen Mal d 1.03D (Mal d 1): from milk: .alpha. lactalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen m 2), tropomyosin fast isoform; from wheat and/or other cerials: high molecular weight glutenin, low molecular weight glutenin, alpha- and gamma gliadin, hordein, secalin, avenin; from strawberry: major strawberry allergy Fra a 1-E (Fra a 1), from banana: profilin (Mus xp 1). A list of other protein allergens is provided at (http://worldwideweb.meduniwien.ac.at/allergens/allfam/).

### Example 8

### Application to Autoimmunity

As outlined herein, PS-binding domains coupled to immunogenic compounds can be used as a tolerizing agent for immunogenic compounds (protein therapeutics). This has implications for the design of protein therapeutics.

Administration of a monoclonal antibody, autologous cytokine, or foreign protein in conjunction with a PSBD will suppress adverse T effector immune responses. In vivo, TRegs act through dendritic cells to limit autoreactive T-cell activation, thus preventing their differentiation and acquisition of effector functions. By limiting the supply of activated pathogenic cells, TRegs prevent or slow down the progression of autoimmune diseases. This protective mechanism appears, however, insufficient in autoimmune individuals, likely because of a shortage of TRegs cells and/or the development and accumulation of TReg-resistant pathogenic T cells over the long disease course. Thus, restoration of self-tolerance in these patients can require purging of pathogenic T cells along with infusion of TRegs with increased ability to control ongoing tissue injury. Organ specific autoimmune conditions, such as thyroiditis and insulin-dependent diabetes mellitus have been attributed to a breakdown of this tolerance mechanism.

Presentation of antigens linked to a PSBD can both induce TRegs and drive the death of pathogenic T cells.

In various alternatives, the antigen in Example 8 can be one or more of the following: proinsulin (diabetes), collagens (rheumatoid arthritis), myelin basic protein (multiple sclerosis). There are many proteins that are human autoimmune proteins, a term referring to various autoimmune diseases wherein the protein or proteins causing the disease are known or can be established by routine testing. Embodiments include testing a patient to identify an autoimmune protein and creating an antigen for use in a molecular fusion and creating immunotolerance to the protein. Embodiments include an antigen, or choosing an antigen from, one or more of the following proteins. In type 1 diabetes mellitus, several main antigens have been identified: insulin, proinsulin, preproinsulin, glutamic acid decarboxylase-65 (GAD-65), GAD-67, insulinoma-associated protein 2 (IA-2), and insulinoma-associated protein 2.beta. (IA-2.beta.); other antigens include ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S100.beta., glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor. In Addison's disease, main antigens include 21 hydroxylase, 17.alpha.-hydroxylase, and P450 side chain cleavage enzyme (P450scc); other antigens include ACTH receptor, P450c21 and P450c17. In premature ovarian failure, main antigens include FSH receptor and .alpha.-enolase. In autoimmune hypophysitis, or pituitary autoimmune disease, main antigens include pituitary gland-specific protein factor (PGSF) 1a and 2; another antigen is type 2 iodothyronine deiodinase. In multiple sclerosis, main antigens include myelin basic protein, myelin oligodendrocyte glycoprotein and proteolipid protein. In rheumatoid arthritis, a main antigen is collagen II. In immunogastritis, a main antigen is H.sup.+, K.sup.+-ATPase. In pernicious angemis, a main antigen is intrinsic factor. In celiac disease, main antigens are tissue transglutaminase and gliadin. In vitiligo, a main antigen is tyrosinase, and tyrosinase related protein 1 and 2. In myasthenia gravis, a main antigen is acetylcholine receptor. In pemphigus vulgaris and variants, main antigens are desmoglein 3, 1 and 4; other antigens include pemphaxin, desmocollins, plakoglobin, perplakin, desmoplakins, and acetylcholine receptor. In bullous pemphigoid, main antigens include BP180 and BP230; other antigens include plectin and laminin 5. In dermatitis herpetiformis Duhring, main antigens include endomysium and tissue transglutaminase. In epidermolysis bullosa acquisita, a main antigen is collagen VII. In systemic sclerosis, main antigens include matrix metalloproteinase 1 and 3, the collagen-specific molecular chaperone heat-shock protein 47, fibrillin-1, and PDGF receptor; other antigens include Scl-70, U1 RNP, Th/To, Ku, Jo1, NAG-2, centromere proteins, topoisomerase I, nucleolar proteins, RNA polymerase I, II and III, PM-Slc, fibrillarin, and B23. In mixed connective tissue disease, a main antigen is U1snRNP, In Sjogren's syndrome, the main antigens are nuclear antigens SS-A and SS-B; other antigens include fodrin, poly(ADP-ribose) polymerase and topoisomerase. In systemic lupus erythematosus, main antigens include nuclear proteins including SS-A, high mobility group box 1 (HMGB1), nucleosomes, histone proteins and double-stranded DNA. In Goodpasture's syndrome, main antigens include glomerular basement membrane proteins including collagen IV. In rheumatic heart disease, a main antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

### Example 9

### Application to Diabetes

Type 1 (Juvenile) diabetes is an organ-specific autoimmune disease resulting from destruction of insulin-producing pancreatic beta-cells. In non-diabetics, islet cell antigen-specific T cells are either deleted in thymic development or are converted to T regulatory cells that actively suppress effector responses to islet cell antigens. In juvenile diabetics and in the NOD mouse model of juvenile diabetes, these tolerance mechanisms are missing. In their absence, islet cell antigens are presented by human leukocyte antigen (HLA) class I and II molecules and are recognized by CD8(+) and CD4(+) autoreactive T cells. Destruction of islet cells by these auto-reactive cells eventually leads to glucose intolerance.

Co-administration of islet cell antigens in association with PSBD will lead to the activation of natural T regulatory cells and the conversion of existing antigen specific effector T cell to a regulatory phenotype. In this way a deleterious autoimmune response is redirected, leading to the induction of antigen-specific adaptive tolerance. Modulation of autoimmune responses to autologous epitopes by induction of antigen specific tolerance can prevent ongoing beta-cell destruction.

Accordingly, the PSBD-linked to one or more of the following antigens will work in methods for the prevention or treatment of diabetes: insulin, proinsulin, preproinsulin, glutamic acid decarboxylase-65 (GAD-65), GAD-67, insulinoma-associated protein 2 (IA-2), and insulinoma-associated protein 2beta (IA-2beta); other antigens include ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S100beta, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5.

### Example 10

### Application to Hepatitus B (HBV) Infection

Chronic HBV is usually either acquired (by maternal fetal transmission) or can be a rare outcome of acute HBV infection in adults. Acute exacerbations of chronic hepatitis B (CH-B) are accompanied by increased cytotoxic T cell responses to hepatitis B core and e antigens (HBcAg/HBeAg). In a recent study, the SYFPEITHI T cell epitope mapping system was used to predict MHC class II-restricted epitope peptides from the HBcAg and HbeAg. MHC class II tetramers using the high scoring peptides were constructed and used to measure TReg and CTL frequencies. The results showed that TReg cells specific for HBcAg declined during exacerbations accompanied by an increase in HBcAg peptide-specific cytotoxic T cells, During the tolerance phase, FOXp3-expressing TReg cell clones were identified.

These data suggest that the decline of HbcAg TRegT cells accounts for the spontaneous exacerbations on the natural history of chronic hepatitis B virus infection.

Accordingly, a PSBD linked to HBV antigens are useful for the prevention or treatment of viral infection, e.g., HBV infection, by promoting the development of HcvAg TRegs. Thus a PSBD linked to HBV core or e antigens (HBcAg or HBeAg) is administered to a subject in need thereof to allow for the treatment and/or prevention of HBV infection.

### Example 11

### Application to SLE

A TReg epitope that plays a role in Systemic Lupus Erythematosis (SLE) or Sjogren's syndrome has been defined. This peptide encompasses residues 131-1S1 (RIHMVYSKRSGKPRGYAFIEY; SEQ ID NO: 1) of a spliceosome protein. Binding assays with soluble HLA class II molecules and molecular modeling experiments indicate that the epitope behaves as promiscuous epitope and binds to a large panel of human DR molecules. In contrast to normal T cells and T cells from non-lupus autoimmune patients, PBMCs from 40% of randomly selected lupus patients contain T cells that proliferate in response to peptide 131-1SI.

Accordingly, a PSBD is co-administered in combination with the epitope from above to a subject at risk of SLE and in turn results in the prevention and/or treatment of SLE.

### Example 12

### Application to Graves' Disease

Graves' disease is an autoimmune disorder that is characterized by antibodies to self-thyroid stimulating hormone receptor (TSHR) leading to leading to hyperthyroidism, or an abnormally strong release of hormones from the thyroid gland. Several genetic factors can influence susceptibility to Graves' disease. Females are much more likely to contract the disease than males; White and Asian populations are at higher risk than black populations and HLA DRB1-0301 is closely associated with the disease.

Accordingly, co-administration of a PS-binding domain with TSHR or other Graves' disease antigens or portions thereof to a subject at risk of Graves' Disease is useful for the prevention or treatment of Graves' disease.

### Example 13

### Application to Autoimmune Thyroiditis

Autoimmune Thyroiditis is a condition that occurs when antibodies arise to self thyroid peroxidase and/or thyroglobulin, which cause the gradual destruction of follicles in the thyroid gland. HLA DRS is closely associated with the disease.

Accordingly, coadministration of a PSBD with thyroid peroxidase and/or thyroglobulin TSHR or portions thereof to a subject at risk of autoimmune thyroiditis is useful for the prevention and/or treatment of autoimmune thyroiditis.

### Example 14

As noted above, type1 diabetes involves inflammation that destroys the beta cells of the pancreas. CD8T cells react to various antigens, including preproinsulin, glutamic acid decarboxylase, IA2, ZnT8 and IGRP. Attempts are being made to limit or prevent disease by expressing versions of these proteins in a way that induces tolerance.

FIG. 3 illustrates one option for doing this utilizing a PSBD linked to human prepro-insulin. Pre-pro-insulin sequence is underlined and the C2 domain of lactadherin is shown in bold (nucleotide sequence—SEQ ID NO: 4; protein sequence—SEQ ID NO: 5), The C2 domain which is sufficient to bind PS with high affinity. Expression of this fusion protein, or, in the alternative, related fusion proteins, which can include a flexible linker between insulin and the PSBD, either from naked DNA, or through vector-based delivery, in the form of an AAV delivered into muscle or some other tissue, can be used to bring about continuous expression of the protein.

Other PSBDs from the list above, or derived through screens, can be used in a similar way, attached through genetic fusion to either the N- or C-terminus of insulin. Note, however, that if the PSBD is attached to the N-terminus a signal sequence for secretion should be provided and the insulin signal sequence should be removed.

### Example 15

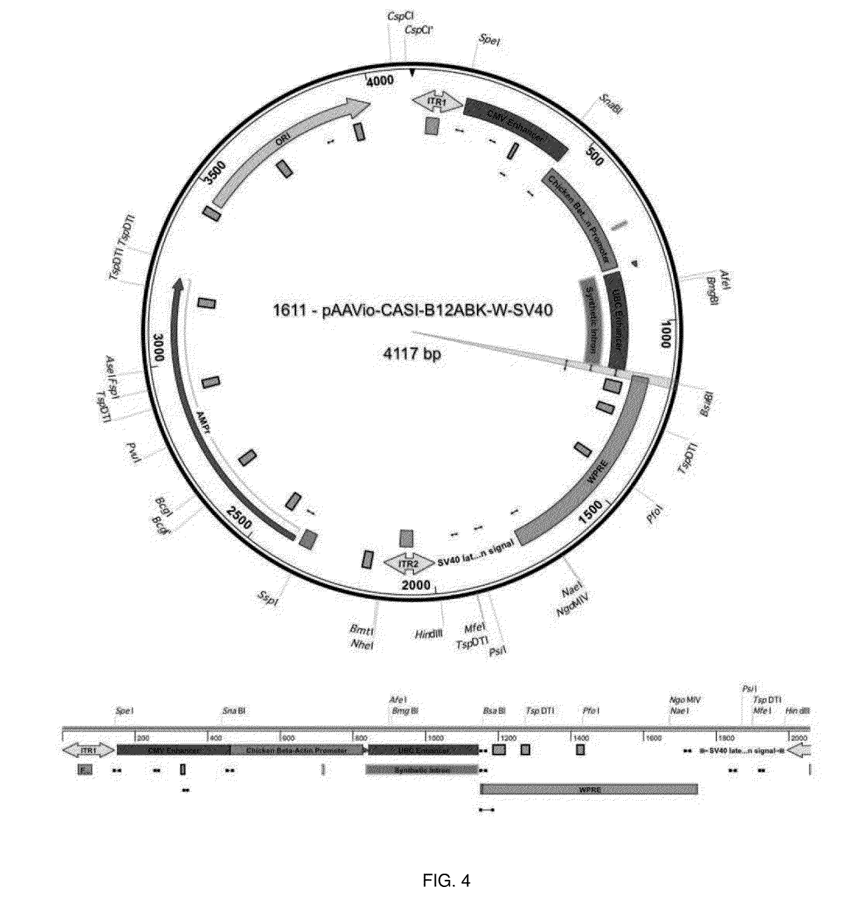

In an implementation involving expression of an antigen-PSBD fusion using AAV, one can use the vector pictured in FIG. 4, in circular and/or linear formats (Balazs et al., 2012). Using this vector, it is possible to drive the expression of milligram per ml levels of antibodies in mice, when viral particles carrying the vector are introduced into skeletal muscle through injection. The gene of interest (which can include a PSBD fusion to an antigen) is inserted downstream of the promoter and upstream of the WRPE motif.

The foregoing description and Examples detail certain specific embodiments of the invention. It will be appreciated, however, that no matter how detailed the foregoing may appear in text, the invention may be practiced in many ways and the invention should be construed in accordance with the appended claims and any equivalents thereof. While the present teachings have been described in terms of these exemplary embodiments, the skilled artisan will readily understand that numerous variations and modifications of these exemplary embodiments are possible without undue experimentation. All such variations and modifications are within the scope of the current teachings. The foregoing examples are provided to better illustrate the disclosed teachings and are not intended to limit the scope of the teachings presented herein. All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls. The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing appears in text, the invention may be practiced in many ways and the invention should be construed in accordance with the appended claims and any equivalents thereof.
Anderson, H. A., Maylock, C. A., Williams, J. A., Paweletz, C, P., Shu, H., and Shacter, E. (2003). Serum-derived protein S binds to phosphatidylserine and stimulates the phagocytosis of apoptotic cells. Nature immunology 4, 87-91.
Balazs, A. B., Chen, J., Hong, C. M., Rao, D. S., Yang, L., and Baltimore, D. (2012). Antibody-based protection against HIV infection by vectored immunoprophylaxis. Nature 481, 81-84,
Battisto, J. R., and Bloom, B. R. (1966). Dual immunological unresponsiveness induced by cell membrane coupled hapten or antigen. Nature 212, 156-157.
Burtea, C., Laurent, S., Lancelot, E., Ballet, S., Murariu, O., Rousseaux, O., Port, M., Vander Elst, L., Corot, C., and Muller, R. N. (2009). Peptidic targeting of phosphatidylserine for the MRI detection of apoptosis in atherosclerotic plaques. Molecular pharmaceutics 6, 1903-1919.
Cremel, M., Guerin, N., Horand, F., Banz, A., and Godfrin, Y. (2013). Red blood cells as innovative antigen carrier to induce specific immune tolerance. International journal of pharmaceutics 443, 39-49.
Dasgupta, S. K., Abdel-Monem, H., Guchhait, P., Nagata, S., and Thiagarajan, P. (2008). Role of lactadherin in the clearance of phosphatidylserine-expressing red blood cells. Transfusion 48, 2370-2376.
Gaitonde, P., Peng, A., Straubinger, R. M., Bankert, R. B., and Balu-Iyer, S. V. (2011). Phosphatidylserine reduces immune response against human recombinant Factor VIII in Hemophilia A mice by regulation of dendritic cell function. Clin Immunol 138, 135-145.
Gaitonde, P., Ramakrishnan, R., Chin, J., Kelleher, R. J., Jr., Bankert, R. B., and Balu-Iyer, S. V. (2013). Exposure to factor VIII protein in the presence of phosphatidylserine induces hypo-responsiveness toward factor VIII challenge in hemophilia A mice. J Biol Chem 288, 17051-17056.
Getts, D. R., McCarthy, D. P., and Miller, S. D. (2013). Exploiting apoptosis for therapeutic tolerance induction. J Immunol 191, 5341-5346.
Getts, D. R., Turley, D. M., Smith, C. E., Harp, C. T., McCarthy, D., Feeney, E. M., Getts, M. T., Martin, A. J., Luo, X., Terry, R. L., et al. (2011). Tolerance induced by apoptotic antigen-coupled leukocytes is induced by PD-L1+ and IL-10-producing splenic macrophages and maintained by T regulatory cells. J Immunol 187, 2405-2417.
Gong, J., Archer, R., Brown, M., Fisher, S., Chang, C., Peacock, M., Hughes, C., and Freimark, B. (2013). Measuring response to therapy by near-infrared imaging of tumors using a phosphatidylserine-targeting antibody fragment. Molecular imaging 12, 244-256.
Goode, I., Xu, H., and Ildstad, S. T. (2014). Regulatory B cells: the new “it” cell. Transplantation proceedings 46, 3-8.
Guillen, K. P., Restuccia, A., Kurkjian, C., and Harrison, R. G. (2015). Annexin V-Directed Enzyme Prodrug Therapy Plus Docetaxel for the Targeted Treatment of Pancreatic Cancer, Pancreas 44, 945-952.
Hanayama, R., Miyasaka, K., Nakaya, M., and Nagata, S. (2006). MFG-E8-dependent clearance of apoptotic cells, and autoimmunity caused by its failure. Current directions in autoimmunity 9, 162-172.
Hanayama, R., Tanaka, M., Miwa, K., Shinohara, A., Iwamatsu, A., and Nagata, S. (2002). Identification of a factor that links apoptotic cells to phagocytes. Nature 417, 182-187.
He, M., Kubo, H., Morimoto, K., Fujino, N., Suzuki, T., Takahasi, T., Yamada, M., Yamaya, M., Maekawa, T., Yamamoto, Y., et al. (2011). Receptor for advanced glycation end products binds to phosphatidylserine and assists in the clearance of apoptotic cells, EMBO Rep 12, 358-364.
Igarashi, K., Asai, K., Kaneda, M., Umeda, M., and Inoue, K. (1995). Specific binding of a synthetic peptide derived from an antibody complementarity determining region to phosphatidylserine. J Biochem 117, 452-457.
Ishimoto, Y., Ohashi, K., Mizuno, K., and Nakano, T. (2000). Promotion of the uptake of PS liposomes and apoptotic cells by a product of growth arrest-specific gene, gash. J Biochem 127, 411-417.
Itoh, S., Yokoyama, R., Murase, C., Takii, T., Tsuji, T., and Onozaki, K. (2012). Staphylococcal superantigen-like protein 10 binds to phosphatidylserine and apoptotic cells. Microbiology and immunology 56, 363-371.
Jefferis, R., and Lefranc, M. P. (2009). Human immunoglobulin allotypes: possible implications for immunogenicity. mAbs 1, 332-338.
Jones, B. S., Lamb, L. S., Goldman, F., and Di Stasi, A. (2014). Improving the safety of cell therapy products by suicide gene transfer. Frontiers in pharmacology 5, 254.
Kay, M. A. (2011). State-of-the-art gene-based therapies: the road ahead. Nat Rev Genet 12, 316-328.
Kim, S., Bae, S. M., Seo, J., Cha, K., Piao, M., Kim, S. J., Son, H. N., Park, R. W., Lee, B. H., and Kim, I. S. (2015). Advantages of the phosphatidylserine-recognizing peptide PSP1 for molecular imaging of tumor apoptosis compared with annexin V. PLoS One 10, e0121171.
Kobayashi, N., Karisola, P., Pena-Cruz, V., Dorfman, D. M., Jinushi, M., Umetsu, S. E., Butte, M. J., Nagumo, H., Chernova, I., Zhu, B., et al. (2007). TIM-1 and TIM-4 glycoproteins bind phosphatidylserine and mediate uptake of apoptotic cells. Immunity 27, 927-940.
Kontos, S., Kourtis, I. C., Dane, K. Y., and Hubbell, J. A. (2013). Engineering antigens for in situ erythrocyte binding induces T-cell deletion. Proc Natl Acad Sci USA 110, E60-68.
Kuriyama, S., Taguchi, Y., Nishimura, K., Yanagibashi, K., Katayama, Y., and Niidoime, T. (2009). Improvement of peptide vectors for gene delivery with active targeting profiles for phosphatidylserine. Journal of peptide science: an official publication of the European Peptide Society 15, 114-119.
Landsteiner, K., and Jacobs, J. (1935). Studies on the Sensitization of Animals with Simple Chemical Compounds. J Exp Med 61, 643-656.
Laumonier, C., Segers, J., Laurent, S., Michel, A., Coppee, F., Belayew, A., Elst, L. V., and Muller, R. N. (2006). A new peptidic vector for molecular imaging of apoptosis, identified by phage display technology. Journal of biomolecular screening 11, 537-545.
Lee, S. J., Park, S. Y., Jung, M. Y., Bae, S. M., and Kim, I. S. (2011). Mechanism for phosphatidylserine-dependent erythrophagocytosis in mouse liver. Blood 117, 5215-5223.
Li, J., Olvera, A. I., Akbari, o. S., Moradian, A., Sweredoski, M. J., Hess, S., and Hay, B. A. (2015). Vectored antibody gene delivery mediates long-term contraception. Current Biology (in press), 123.
Lucas, N., and Cho, W. (2011). Phosphatidylserine binding is essential for plasma membrane recruitment and signaling function of 3-phosphoinositide-dependent kinase-1. J Biol Chem 286, 41265-41272.
Luo, X., Pothoven, K. L., McCarthy, D., DeGutes, M., Martin, A., Getts, D. R., Xia, G., He, J., Zhang, X., Kaufman, D. B., et al. (2008). ECDI-fixed allogeneic splenocytes induce donor-specific tolerance for long-term survival of islet transplants via two distinct mechanisms. Proc Natl Acad Sci USA 105, 14527-14532.
Miller, S. D., Wetzig, R. P., and Claman, H. N. (1979). The induction of cell-mediated immunity and tolerance with protein antigens coupled to syngeneic lymphoid cells. J Exp Med 149, 758-773.
Miyake, Y., Asano, K., Kaise, H., Uemura, M., Nakayama, M., and Tanaka, M. (2007). Critical role of macrophages in the marginal zone in the suppression of immune responses to apoptotic cell-associated antigens. J Clin Invest 117, 2268-2278,
Miyanishi, M., Tada, K., Koike, M., Uchiyama, Y., Kitamura, T., and Nagata, S. (2007). Identification of Tim4 as a phosphatidylserine receptor. Nature 450, 435-439.
Morizono, K., Xie, Y., Olafsen, T., Lee, B., Dasgupta, A., Wu, A. M., and Chen, I. S. (2011). The soluble serum protein Gas6 bridges virion envelope phosphatidylserine to the TAM receptor tyrosine kinase Ax1 to mediate viral entry. Cell Host Microbe 9, 286-298.
Morton, L. A., Yang, H., Saludes, J. P., Fiorini, Z., Beninson, L., Chapman, E. R., Fleshner, M., Xue, D., and Yin, H. (2013). MARCKS-ED peptide as a curvature and lipid sensor. ACS chemical biology 8, 218-225.
Nagata, S., Hanayama, R., and Kawane, K. (2010). Autoimmunity and the clearance of dead cells. Cell 140, 619-630.
Nakahashi-Oda, C., Tahara-Hanaoka, S., Honda, S., Shibuya, K., and Shibuya, A. (2012). Identification of phosphatidylserine as a ligand for the CD300a immunoreceptor. Biochem Biophys Res Commun 417, 646-650.
Neves, A. A., and Brindle, K. M. (2014). Imaging cell death. Journal of nuclear medicine: official publication, Society of Nuclear Medicine 55, 1-4,
Paidassi, H., Tacnet-Delorme, P., Garlatti, V., Darnault, C., Ghebrehiwet, B., Gaboriaud, C., Arlaud, G. J., and Frachet, P. (2008). C1q binds phosphatidylserine and likely acts as a multiligand-bridging molecule in apoptotic cell recognition. J Immunol 180, 2329-2338.
Pandey, J. P., and Li, Z. (2013). The forgotten tale of immunoglobulin allotypes in cancer risk and treatment. Experimental hematology & oncology 2, 6.
Park, S. Y., Jung, M. Y., Kim, H. J., Lee, S. J., Kim, S. Y., Lee, B. H., Kwon, T. H., Park, R. W., and Kim, I. S. (2008a). Rapid cell corpse clearance by stabilin-2, a membrane phosphatidylserine receptor. Cell Death Differ 15, 192-201.
Park, S. Y., Kim, S. Y., Jung, M. Y., Bae, D. J., and Kim, I. S. (2008b). Epidermal growth factor-like domain repeat of stabilin-2 recognizes phosphatidylserine during cell corpse clearance. Mol Cell Biol 28, 5288-5298.
Purohit, V. S., Ramani, K., Sarkar, R., Kazazian, H. H., Jr., and Balasubramanian, S. V. (2005). Lower inhibitor development in hemophilia A mice following administration of recombinant factor VIII-O-phospho-L-serine complex. J Biol Chem 280, 17593-17600.
Qi, X., and Grabowski, G. A. (2001). Differential membrane interactions of saposins A and C: implications for the functional specificity. J Biol Chem 276, 27010-27017.
Qiu, F., Hu, M., Tang, B., Liu, X., Zhuang, H., Yang, J., and Hua, Z. C. (2013). Annexin V-TRAIL fusion protein is a more sensitive and potent apoptotic inducer for cancer therapy. Scientific reports 3, 3565.
Ramani, K., Miclea, R. D., Purohit, V. S., Mager, D. E., Straubinger, R. M., and Balu-Iyer, S. V. (2008). Phosphatidylserine containing liposomes reduce immunogenicity of recombinant human factor VIII (rFVIII) in a murine model of hemophilia A. Journal of pharmaceutical sciences 97, 1386-1398.
Ravishankar, B., and McGaha, T. L. (2013). O death where is thy sting? Immunologic tolerance to apoptotic self. Cell Mol Life Sci 70, 3571-3589.
Ravishankar, B., Shinde, R., Liu, H., Chaudhary, K., Bradley, J., Lemos, H. P., Chandler, P., Tanaka, M., Munn, D. H., Mellor, A. L., et al. (2014). Marginal zone CD169+ macrophages coordinate apoptotic cell-driven cellular recruitment and tolerance. Proc Natl Acad Sci USA 111, 4215-4220.
Reddy Nanga, R. P., Vivekanandan, S., and Yoon, H. S. (2007). Expression, purification and characterization of C2 domain of milk fat globule-EGF-factor 8-L. Protein expression and purification 52, 329-333.
Rosenbaum, S., Kreft, S., Etich, J., Frie, C., Stermann, J., Grskovic, I., Frey, B., Mielenz, D., Poschl, E., Gaipl, U., et al. (2011). Identification of novel binding partners (annexins) for the cell death signal phosphatidylserine and definition of their recognition motif. J Biol Chem 286, 5708-5716.
Santiago, C., Ballesteros, A., Martinez-Munoz, L., Mellado, M., Kaplan, G. G., Freeman, G. J., and Casasnovas, J. M. (2007). Structures of T cell immunoglobulin mucin protein 4 show a metal-Ion-dependent ligand binding site where phosphatidylserine binds. Immunity 27, 941-951.
Schweigert, O., Dewitz, C., Moller-Hackbarth, K., Trad, A., Garbers, C., Rose-John, S., and Scheller, J. (2014). Soluble T cell immunoglobulin and mucin domain (TIM)-1 and -4 generated by A Disintegrin And Metalloprotease (ADAM)-10 and -17 bind to phosphatidylserine. Biochim Biophys Acta 1843, 275-287.
Shao, C., Novakovic, V. A., Head, J. F., Seaton, B. A., and Gilbert, G. E. (2008). Crystal structure of lactadherin C2 domain at 1.7A resolution with mutational and computational analyses of its membrane-binding motif. J Biol Chem 283, 7230-7241.
Simhadri, V. R., Andersen, J. F., Calvo, E., Choi, S. C., Coligan, J. E., and Borrego, F. (2012). Human CD300a binds to phosphatidylethanolamine and phosphatidylserine, and modulates the phagocytosis of dead cells. Blood 119, 2799-2809.
Srivastava, A., Quinn-Allen, M. A., Kim, S. W., Kane, W. H., and Lentz, B. R. (2001). Soluble phosphatidylserine binds to a single identified site in the C2 domain of human factor Va. Biochemistry 40, 8246-8255.
Steinman, R. M., and Nussenzweig, M. C. (2002). Avoiding horror autotoxicus: the importance of dendritic cells in peripheral T cell tolerance. Proc Natl Acad Sci USA 99, 351-358.
Steinman, R. M., Turley, S., Mellman, I., and Inaba, K. (2000). The induction of tolerance by dendritic cells that have captured apoptotic cells. J Exp Med 191, 411-416.
Sun, E., Gao, Y., Chen, J., Roberts, A. I., Wang, X., Chen, Z., and Shi, Y. (2004). Allograft tolerance induced by donor apoptotic lymphocytes requires phagocytosis in the recipient. Cell Death Differ 11, 1258-1264.
Thapa, N., Kim, S., So, I. S., Lee, B. H., Kwon, I. C., Choi, K., and Kim, I. S. (2008). Discovery of a phosphatidylserine-recognizing peptide and its utility in molecular imaging of tumour apoptosis. J Cell Mol Med 12, 1649-1660.
Tietjen, G. T., Gong, Z., Chen, C. H., Vargas, E., Crooks, J. E., Cao, K. D., Heffern, C. T., Henderson, J. M., Meron, M., Lin, B., et al. (2014). Molecular mechanism for differential recognition of membrane phosphatidylserine by the immune regulatory receptor Tim4. Proc Natl Acad Sci USA 111, E1463-1472.
Tung, T. T., Nagaosa, K., Fujita, Y., Kita, A., Mori, H., Okada, R., Nonaka, S., and Nakanishi, Y. (2013). Phosphatidylserine recognition and induction of apoptotic cell clearance by Drosophila engulfment receptor Draper. J Biochem 153, 483-491.
Turley, D. M., and Miller, S. D. (2007). Peripheral tolerance induction using ethylenecarbodiimide-fixed APCs uses both direct and indirect mechanisms of antigen presentation for prevention of experimental autoimmune encephalomyelitis. J Immunol 178, 2212-2220.
Wang, P., and Zheng, S. G. (2013). Regulatory T cells and B cells: implication on autoimmune diseases. International journal of clinical and experimental pathology 6, 2668-2674.
Wang, Y., and Wang, Z. (2015). Efficient backsplicing produces translatable circular mRNAs. RNA 21, 172-179.
Xiong, C., Brewer, K., Song, S., Zhang, R., Lu, W., Wen, X., and Li, C. (2011). Peptide-based imaging agents targeting phosphatidylserine for the detection of apoptosis. Journal of medicinal chemistry 54, 1825-1835.
Yang, H., Chen, Y. Z., Zhang, Y., Wang, X., Zhao, X., Godfroy, J. I., 3rd, Liang, Q., Zhang, M., Zhang, T., Yuan, Q., et al. (2015). A lysine-rich motif in the phosphatidylserine receptor PSR-1 mediates recognition and removal of apoptotic cells. Nature communications 6, 5717,
Ye, H., Li, B., Subramanian, V., Choi, B. H., Liang, Y., Harikishore, A., Chakraborty, G., Baek, K., and Yoon, H. S. (2013). NMR solution structure of C2 domain of MFG-E8 and insights into its molecular recognition with phosphatidylserine. Biochim Biophys Acta 1828, 1083-1093.
Yin, H., Kanasty, R. L., Eltoukhy, A. A., Vegas, A. J., Dorkin, J. R., and Anderson, D. G. (2014). Non-viral vectors for gene-based therapy. Nat Rev Genet 15, 541-555.
Youn, H., and Chung, J. K. (2015). Modified mRNA as an alternative to plasmid DNA (pDNA) for transcript replacement and vaccination therapy. Expert opinion on biological therapy 15, 1337-1348,
Zeng, W., Wang, X., Xu, P., Liu, G., Eden, H. S., and Chen, X. (2015). Molecular imaging of apoptosis: from micro to macro. Theranostics 5, 559-582.

1. A tolerance-inducing molecule, said tolerance-inducing molecule comprising:
an antigen; and
a phosophatidylserine-binding protein associated with the antigen to form an antigen-phosophatidylserine-binding protein fusion (“APBP”) and/or an antigen-phosophatidylserine-binding protein complex (“APBC”).

2. The tolerance-inducing molecule of claim 1, wherein the antigen comprises a therapeutic molecule.

3. The tolerance-inducing molecule of claim 2, wherein the therapeutic molecule comprises a protein.

4. The tolerance-inducing molecule of claim 3, wherein the protein comprises a therapeutic antibody, a therapeutic enzyme, a blood coagulation factor, a therapeutic cofactor, an allergen, proteins deficient by genetic disease, proteins with non-human glycosylation, proteins with a glycosylation pattern not present in the relevant species, non-human proteins, non-native proteins, synthetic proteins not normally found in the species of interest, human food antigens, human transplantation antigens, human autoimmune antigens, and/or normally-occurring self antigens to which an immune response is initiated in autoimmune disease.

5. The tolerance-inducing molecule of claim 4, wherein the phosophatidylserine-binding protein is covalently linked to the antigen, to form an APBP.

6. The tolerance-inducing molecule of claim 5, wherein the phosophatidylserine-binding protein is directly linked to the antigen.

7. The tolerance-inducing molecule of claim 5, wherein the phosophatidylserine-binding protein is indirectly linked to the antigen via a linker.

8. The tolerance-inducing molecule of claim 7, wherein the linker comprises a chemical linker or a peptide linker.

9. The tolerance-inducing molecule of claim 7, wherein the linker is genetically encoded.

10. The tolerance-inducing molecule of claim 4, wherein the phosophatidylserine-binding protein is non-covalently linked to the antigen, to form an APBC.

11. The tolerance-inducing molecule of claim 4, wherein the protein comprises a therapeutic antibody.

12. The tolerance-inducing molecule of claim 1, wherein the protein comprises a therapeutic antibody

13. The tolerance-inducing molecule of claim 1, wherein the protein comprises a blood coagulation factor.

14. The tolerance-inducing molecule of claim 1, wherein the phosophatidylserine-binding protein comprises a PS-binding domain.

15. The tolerance-inducing molecule of claim 1, wherein the phosophatidylserine-binding protein comprises at least a binding domain of at least one of: Tim1-4 proteins, Lactadherin/MFG-E8, Stabilin-2, Gas6/protein S, C300a, BAI1, RAGE, PDK1, Annexin1-5, C1Q, Factor V, Drosophila Draper, or Stapylococcal SSL10, PSR-1, the peptides LSYYPSYC (SEQ ID NO: 6), AREDGYDGAMDY (SEQ ID NO: 7), LIKKPF (SEQ ID NO: 8), CLIKKPF (SEQ ID NO: 9), PGDLSR (SEQ ID NO: 10), CPGDLSR (SEQ ID NO: 11), FNFRLKAGQKIRFG (SEQ ID NO: 12), FNFRLKAGAKIRFG (SEQ ID NO: 13), FNFRLKVGAKIRFG (SEQ ID NO: 14), FNFRLKTGAKIRFG (SEQ ID NO: 15), FNFRLKCGAKIRFG (SEQ ID NO: 16), RSRRMTRRARAA (SEQ ID NO: 17), TLVSSL (SEQ ID NO: 18), TRYLRIHPRSWVHQIALRLRYLRIHPRSWVHQIALRS (SEQ ID NO: 19), TRYLRLHPRSWVHQLALRLRYLRLHPRSWVHQLALRS (SEQ ID NO: 20), KKKKRFSFKKSFKLSGFSFKKNKK (SEQ ID NO: 21), saposin C, or phosphatidylserine-binding monoclonal antibodies.

16. The tolerance-inducing molecule of claim 1, wherein the antigen comprises a HBV antigen.

17. The tolerance-inducing molecule of claim 1, wherein the antigen comprises RIHMVYSKRSGKPRGYAFIEY (SEQ ID NO: 1).

18. A nucleic acid sequence encoding any one or more of the tolerance-inducing molecules of claim 1.

19. A vector comprising the nucleic acid sequence of claim 18.

20. A composition comprising a mixture of any one of the tolerance-inducing molecules of claim 1 and a free therapeutic molecule, wherein the free therapeutic molecule is not associated with the antigen.

21. The composition of claim 20, wherein the tolerance-inducing molecule is present in a first amount and the free therapeutic molecule is present in a second amount.

22. The composition of claim 20, wherein the first amount is less than the second amount.

23. The composition of claim 20, wherein the first amount is about the same as the second amount.

24. The composition of claim 20, wherein the first amount is more than the second amount.

25. The composition of claim 20, wherein the antigen of the tolerance-inducing molecule is the same type of molecule as the free therapeutic molecule.

26. The composition of claim 20, wherein the antigen of the tolerance-inducing molecule and the free therapeutic molecule are both therapeutic molecules.

27. The composition of claim 20, wherein the antigen of the tolerance-inducing molecule and the free therapeutic molecule are both proteins.

28. The composition of claim 20, wherein the antigen of the tolerance-inducing molecule and the free therapeutic molecule are both at least one of: a therapeutic antibody, a therapeutic enzyme, a blood coagulation factor, a therapeutic cofactor, an allergen, a protein deficient by genetic disease, a protein with non-human glycosylation, a non-native protein, a protein having a glycosylation pattern not present in a species, a non-human protein, a non-native protein, a synthetic protein, a recombinant protein, a human food antigen, a human transplantation antigen, a human autoimmune antigen, an antigen to which an immune response is initiated in autoimmune disease, insulin, proinsulin, preproinsulin, glutamic acid decarboxylase-65 (GAD-65), GAD-67, insulinoma-associated protein 2 (IA-2), insulinoma-associated protein 2beta (IA-213), ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S100beta, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5; b) thyroglobulin (TG), thyroid peroxidase (TPO), thyrotropin receptor (TSHR), sodium iodine symporter (NIS) and megalin; c) thyroglobulin (TG), thyroid peroxidase (TPO), thyrotropin receptor (TSHR), sodium iodine symporter (NIS), megalin, and insulin-like growth factor 1 receptor; d) calcium sensitive receptor; e) 21-hydroxylase, 17alpha-hydroxylase, P450 side chain cleavage enzyme (P450scc), ACTH receptor, P450c21 and P450c17; f) FSH receptor and .alpha. enolase; g) pituitary gland-specific protein factor (PGSF) 1a, PGSF 2, and type 2 iodothyronine deiodinase; h) myelin basic protein, myelin oligodendrocyte glycoprotein and proteolipid protein; i) collagen II; j) Rsup.+,K.sup.+-ATPase; k) intrinsic factor; l) tissue transglutaminase and gliadin; m) tyrosinase, and tyrosinase related protein 1 and 2; n) acetylcholine receptor; o) desmoglein 3, desmoglein 1, desmoglein 4, pemphaxin, desmocollins, plakoglobin, perplakin, desmoplakins, and acetylcholine receptor; p) BP180, BP230, plectin and laminin 5; q) endomysium and tissue transglutaminase; r) collagen VII; s) matrix metalloproteinase 1 and 3, the collagenspecific molecular chaperone heat-shock protein 47, fibrillin-1, PDGF receptor, Scl-70, U1 RNP, Th/To, Ku, Jo1, NAG-2, centromere proteins, topoisomerase I, nucleolar proteins, RNA polymerase I, II and III, PM-Slc, fibrillarin, and B23; t) U1snRNP; u) SS-A, SS-B, fodrin, poly(ADP-ribose) polymerase, and topoisomerase v) SS-A, high mobility group box 1 (HMGB1), nucleosomes, histone proteins and double-stranded DNA; w) glomerular basement membrane proteins including collagen IV; x) cardiac myosin; and y) aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega; z) antithrombin-III, protein C, factor VIII, factor IX, growth hormone, somatotropin, insulin, pramlintide acetate, mecasermin (IGF-1), beta-gluco cerebrosidase, alglucosidase-.alpha., laronidase (alpha Liduronidase), idursuphase (iduronate-2-sulphatase), galsulphase, agalsidase-beta (alpha-galactosidase), alpha-1 proteinase inhibitor, and albumin; aa) adenosine deaminase, pancreatic lipase, pancreatic amylase, lactase, botulinum toxin type A, botulinum toxin type B, collagenase, hyaluronidase, papain, L-Asparaginase, uricase, lepirudin, streptokinase, anistreplase (anisoylated plasminogen streptokinase activator complex), antithymocyte globulin, crotalidae polyvalent immune Fab, digoxin immune serum Fab, L-arginase, and L methionase; bb) conarachin (Ara h 1), allergen II (Ara h 2), arachis agglutinin, conglutin (Ara h 6), 31 kda major allergen/disease resistance protein homolog (Mal d 2), lipid transfer protein precursor (Mal d 3), major allergen Mal d 1.03D (Mal d 1), alpha lactalbumin (ALA), lactotransferrin, actinidin (Act c 1, Act d 1), phytocystatin, thaumatin-like protein (Act d 2), kiwellin (Act d 5), 2S albumin (Sin a 1), 11S globulin (Sin a 2), lipid transfer protein (Sin a 3), profilin (Sin a 4), profilin (Api g 4), high molecular weight glycoprotein (Api g 5), Pen a 1 allergen (Pen a 1), allergen Pen m 2 (Pen m 2), tropomyosin fast isoform, high molecular weight glutenin, low molecular weight glutenin, alpha- and gamma-gliadin, hordein, secalin, avenin, major strawberry allergy Fra a 1-E (Fra a 1), and profilin (Mus xp 1); and cc) subunits of MHC class I and MHC class II haplotype proteins, and single-amino-acid polymorphisms on minor blood group antigens including RhCE, Kell, Kidd, Duffy and Ss.

29. A method of providing immunological tolerance to an antigen, the method comprising administering an effective amount of a tolerance-inducing molecule, the tolerance-inducing molecule comprising a phosophatidylserine-binding protein that is associated with an antigen to a subject.

30. The method of claim 28, wherein administering comprises oral, intranasal, intramuscular, parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

31. The method of claim 28, wherein administering comprises infusion of the tolerance-inducing molecule.

32. The method of claim 30, wherein infusion comprises an IV injection.

33. The method of claim 28, wherein administering comprises vectored expression of the tolerance-inducing molecule in the subject.

34. The method of claim 28, wherein the subject is to receive a free therapeutic molecule, wherein the free therapeutic molecule is the same type of molecule as the antigen.

35. The method of claim 28, wherein the tolerance is for a treatment of an autoimmune disease.

36. The method of claim 28, wherein the tolerance is for a treatment of asthma.

37. The method of claim 28, wherein the tolerance is for a treatment of an allergy.

38. The method of claim 28, wherein the tolerance is to reduce a risk of a protein-based therapeutic failure due to a host response against the antigen.

39. The method of claim 28, wherein the tolerance is long-term antigen-specific immune tolerance.

40. The method of claim 28, wherein the antigen comprises a therapeutic antibody.

41. The method of claim 28, wherein the antigen comprises a blood coagulation factor.

42. The method of claim 28, wherein the phosophatidylserine-binding protein comprises at least a binding domain of at least one of: Tim1-4 protein, Lactadherin/MFG-E8, Stabilin-2, Gas6/protein S, C300a, BAIL RAGE, PDK1, Annexins, C1Q, Factor V in thrombin cascade, Drosophila Draper, or Stapylococcal SSL10, PSR-1, the peptides CLSYYPSYC (SEQ ID NO: 22), AREDGYDGAMDY (SEQ ID NO: 7), LIKKPF (SEQ ID NO: 8), CLIKKPF (SEQ ID NO: 9), PGDLSR (SEQ ID NO: 10), CPGDLSR (SEQ ID NO: 11), FNFRLKAGQKIRFG (SEQ ID NO: 12), FNFRLKAGAKIRFG (SEQ ID NO: 13), FNFRLKVGAKIRFG (SEQ ID NO: 14), FNFRLKTGAKIRFG (SEQ ID NO: 15), FNFRLKCGAKIRFG (SEQ ID NO: 16), RSRRMTRRARAA (SEQ ID NO: 17), TLVSSL (SEQ ID NO: 18), TRYLRIHPRSWVHQIALRLRYLRIHPRSWVHQIALRS (SEQ ID NO: 19), TRYLRLHPRSWVHQLALRLRYLRLHPRSWVHQLALRS (SEQ ID NO: 20), KKKKRFSFKKSFKLSGFSFKKNKK (SEQ ID NO: 21), saposin C, and phosphatidylserine-binding monoclonal antibodies.

43. The method of claim 28, wherein the subject has or is at risk of at least one of the following: Factor VIII deficiency, an autoimmune disease, type 1 diabetes, multiple sclerosis, lupus, rheumatoid arthritis; a transplant related disorder, graft vs. host disease (GVHD), allergic reaction; immune rejection of biologic medicines including: monoclonal antibodies, replacement proteins including FVIII and/or insulin, a therapeutic toxin, including Botulinum toxin; and the management of immune response to infectious disease.

44. The method of claim 27, wherein the subject is to receive an antibody, a recombinant protein, or a foreign protein.
